(19) United States
(12) Patent Application Publication
(10) Pub. No.: US 2016/0199396 A1
(43) Pub. Date: Jul. 14, 2016

(54) UNIT DOSAGE FORM COMPRISING EMTRICITABINE, TENOFOVIR, DARUNAVIR AND RITONAVIR AND A MONOLITHIC TABLET COMPRISING DARUNAVIR AND RITONAVIR

(71) Applicant: TEVA Pharmaceutical Industries Ltd., Petach Tikva (IL)

(72) Inventors: Nitzan SHAHAR, Kibbutz Tel-Yitzhak (IL); Elina HARONSKY, Rosh Ha'ain (IL); Julia HRAKOVSKY, Rosh Ha-Ayin (IL)

(73) Assignee: TEVA Pharmaceutical Industries Ltd., Petach Tikva (IL)

(21) Appl. No.: 15/051,381

(22) Filed: Feb. 23, 2016

Related U.S. Application Data
(63) Continuation-in-part of application No. PCT/IB2014/001637, filed on Aug. 28, 2014.
(60) Provisional application No. 61/871,674, filed on Aug. 29, 2013.

(51) Int. Cl. A61K 31/675; A61K 9/20; A61K 31/427; A61K 9/28; A61K 31/513; A61K 31/635

(52) U.S. Cl. CPC A61K 31/675; A61K 31/513; A61K 31/635; A61K 31/427; A61K 9/28; A61K 9/20; A61K 9/2095

(57) ABSTRACT

The present invention relates to an oral unit dosage form comprising Emtricitabine, Tenofovir, Darunavir and Ritonavir and a monolithic tablet comprising Darunavir and Ritonavir and their use to treat HIV infection.

### CROSS-REFERENCE TO RELATED APPLICATIONS

This application is a continuation-in-part of International Application No. PCT/IB2014/001637, filed 28 Aug. 2014 and claiming priority from U.S. Provisional Application No. 61/871,674, filed 29 Aug. 2013, and a continuation-in-part of International Application No. PCT/IB2015/052493, filed 7 Apr. 2015 and claiming priority from U.S. Provisional Application No. 61/976,917, filed 8 Apr. 2014, and U.S. Provisional Application No. 62/119,520, filed 23 Feb. 2015. The entire disclosure of each of these applications is incorporated herein by reference for all purposes.

### FIELD OF THE INVENTION

The present invention relates to an oral unit dosage form comprising as active agents, emtricitabine, tenofovir, darunavir and ritonavir and to a monolithic tablet comprising as active agents, darunavir and ritonavir and their use to treat HIV infection. The invention further relates to methods of preparing the oral dosage forms containing the above pharmaceutically active agents.

### BACKGROUND OF THE INVENTION

Tenofovir, the systematic chemical name for which is ({[(2R)-1-(6-amino-9H-purin-9-yl) propan-2-yl]oxy}methyl) phosphonic acid, is a nucleoside reverse transcriptase inhibitor (NRTI) which is used to treat infection by HIV-I. Its synthesis, analogs, formulation and use are described in various publications including, inter alia, U.S. Pat. Nos. 5,922,695; 5,935,946, 5,977,089 and 6,043,230.

Pharmaceutical formulations comprising combinations of tenofovir and another non-nucleoside reverse transcriptase inhibitor (NNRTI), or a protease inhibitor (PI) are described in various publications including, inter alia, U.S. Pat. No. 8,592,397, U.S. Pat. No. 8,716,264 and US20140037732.

Tenofovir disoproxil fumarate (TDF) is a prodrug form of tenofovir. TDF is marketed by Gilead Sciences under the trade name VIREAD®. VIREAD tablets are available in strengths of 150, 200, 250, and 300 mg of tenofovir disoproxil fumarate, which are equivalent to 123, 163, 204 and 245 mg of tenofovir disoproxil, respectively. The tablet also includes the following inactive ingredients: croscarmellose sodium, lactose monohydrate, magnesium stearate, microcrystalline cellulose, and pregelatinized starch.

Tenofovir is also available in a fixed-dose combination with emtricitabine in a product with the brand name TRUVADA®, which has been approved for once-a-day dosing. Each TRUVADA tablet contains 200 mg of emtricitabine and 300 mg of tenofovir disoproxil fumarate, (which is equivalent to 245 mg of tenofovir disoproxil), as active ingredients. The tablets also include the following inactive ingredients: croscarmellose sodium, lactose monohydrate, magnesium stearate, microcrystalline cellulose, and pregelatinized starch. The total weight of the TRUVADA tablet is 1045 mg, having the dimensions of 19 mm×8.5 mm.

Emtricitabine, the systematic chemical name for which is 4-amino-5-fluoro-1-[2-(hydroxymethyl)-1, 3-oxathiolan-5-yl]-pyrimidin-2-one, is another nucleoside reverse transcriptase inhibitor (NRTI) which is used to treat infection by HIV-I. Its synthesis and use are described in various publications including, inter alia, U.S. Pat. Nos. 5,210,085; 5,814,639; 5,914,331, 6, 642, 245 and 7,402,588.

Emtricitabine (FTC) is marketed by Gilead Sciences under the trade name EMTRIVA®. EMTRIVA is available as capsules or as an oral solution. Each capsule contains 200 mg of emtricitabine and also the following inactive ingredients: crospovidone, magnesium stearate, microcrystalline cellulose, povidone, titanium dioxide, gelatin, and FD&C blue No. 2.

Darunavir, the systematic name for which is [(1R,5S,6R)-2,8-dioxabicyclo[3.3.0]oct-6-yl]N-[(2S,3R)-4-[(4-aminophenyl)sulfonyl-(2-methylpropyl)amino]-3-hydroxy-1-phenyl-butan-2-yl]carbamate, is another antiretroviral drug of the protease inhibitor (PI) class which is used to treat HIV-I. Its synthesis, use, salts, formulation and combinations thereof are described in various publications including U.S. Pat. Nos. 6,335,460; 6,248,775, 5,843,946, USRE43596 and WO2013004816.

Darunavir is marketed by Tibotec (Janssen) under the trade name Prezista®. Prezista® tablets are available in strengths of 75 mg, 150 mg, 400 mg, 600 mg, and 800 mg. Each tablet also contains the inactive ingredients colloidal silicon dioxide, crospovidone, magnesium stearate, and microcrystalline cellulose. The 800 mg tablet also contains hypromellose. The total weight of the Prezista® 800 mg tablet is 1048 mg, having the dimensions of 20 mm×8 mm.

Ritonavir, the systematic chemical name for which is 1,3-thiazol-5-ylmethyl N-[(2S,3S,5S)-3-hydroxy-5-[(2S)-3-methyl-2-{[methyl({[2-(propan-2-yl)-1,3-thiazol-4-yl]methyl})carbamoyl]amino}butanamido]-1,6-diphenylhexan-2-yl]carbamate, is another antiretroviral drug of the protease inhibitor (PI) class which is used to treat infection by HIV-I. Its synthesis use, formulation and combinations thereof are described in various publications including, inter alia, U.S. Pat. Nos. 5,541,206; 5,648,497, 6,037,157, 7,364,752 and U.S. Pat. No. 8,268,349.

Ritonavir is marketed by Abbott Laboratories under the trade name Norvir®. Norvir® is available as 100 mg tablet or capsule and as an 80 mg/ml oral solution. Ritonavir is a BCS (biopharmaceutical classification system) class IV material. Thus, ritonavir has very low solubility and permeability. Abbott developed Norvir® tablet, using an amorphous ritonavir in a hot melt extrusion manufacturing process, with a high amount of polymer (NDA 22-417—chemistry review, http://www.accessdata.fda.gov/drugsatfda_docs/nda/2010/022417s000_ChemR.pdf). The Norvir® tablet contains: 100 mg ritonavir, copovidone, sorbitan laurate, calcium hydrogen phosphate anhydrous, anhydrous colloidal silica, sodium stearyl fumarate and is coated with a film-coating formed of hypromellose, titanium dioxide (E171), macrogols, hydroxypropyl cellulose, talc, anhydrous colloidal silica and polysorbate 80. The total weight of the Norvir® tablet is 800 mg, and the tablet has dimensions of 17.2×5.8 mm.

Administered alone, most antiretroviral agents have proved only partially effective, often unable to block HIV replication sufficient to obtain an optimal reduction in viral load or prevent its rise.

To overcome this deficiency, highly active antiretroviral therapy (HAART) has been developed over the years. HAART consists of the co-administration of three antiretroviral agents. The three drugs may be administered separately or administered as a unit dosage form containing three active ingredients.

An intermittent dosage regime comprising the administration of three or four active ingredients, for the treatment of human immunodeficiency virus (HIV) in humans, was suggested in WO 2011/061302 and WO 2011/061303. The manufacture of triple fixed dose combinations is disclosed in WO 1996/030025, WO 2006/135933, and WO2014/184553.

WO2009/081174 describes a dual combination formulation of ritonavir and darunavir; wherein ritonavir is in a first layer and darunavir is in a second layer. According to this disclosure, ritonavir and darunavir, when admixed, results in incompatibilities in which the stability of the active agents is compromised. Accordingly, WO2009/081174 discloses that the two active agents must be separated from each other, i.e., by the provision of a composition in which each agent is present in separate layers. Nevertheless, from the point of view of ease of manufacture, monolithic compositions are generally preferred over multilayer compositions. However, the successful formulation of monolithic dosage forms, is dependent on ensuring that the stability of the active agents in the dosage form is not compromised, as well as ensuring that the dosage form is of a size that enables it to be easily administered.

WO2013057469 describes a combination composition in a kit form. The kit can comprise separate unit dosage forms of various antiretroviral drugs with a set of instructions for their administration. Nevertheless, the administration of separate dosage forms in accordance with a set of instructions does not provide an optimal improvement of patient compliance, especially if the dosage forms are to be taken at different times. Moreover, the instructions may be misplaced, or may be incorrectly followed by the patient.

There is thus a need to provide a chemically stable dosage form containing ritonavir and darunavir which has the advantage of ease of manufacture. There is an additional need to provide dosage forms in which combinations of antiretroviral drugs such as emtricitabine, tenofovir, darunavir and ritonavir, are provided in a composition that can be readily administered so as to further improve patient compliance. In particular, there is a need for less onerous dosage regimen, such as once daily oral dosing, optimally, in a one pill, having the required stability and bioavailability.

The present invention addresses for the first time the preparation of a unit dose form containing emtricitabine, tenofovir, darunavir and ritonavir and of a monolithic tablet containing darunavir and ritonavir.

### SUMMARY OF THE INVENTION

In one aspect, the present invention provides a pharmaceutical formulation in a unit dosage form comprising:
(a) tenofovir or a physiologically functional derivative thereof,
(b) emtricitabine or a physiologically functional derivative thereof,
(c) darunavir or a physiologically functional derivative thereof, and
(d) ritonavir or a physiologically functional derivative thereof; wherein the amount of ritonavir is less than about 100 mg.

In one aspect, the present invention further provides a monolithic tablet comprising:
(a) tenofovir or a physiologically functional derivative thereof,
(b) emtricitabine or a physiologically functional derivative thereof,
(c) darunavir or a physiologically functional derivative thereof and
(d) ritonavir or a physiologically functional derivative thereof, wherein the amount of ritonavir is less than or equal to about 70 mg.

In one aspect, the present invention further provides a monolithic tablet comprising:
(a) tenofovir or a physiologically functional derivative thereof,
(b) emtricitabine or a physiologically functional derivative thereof,
(c) darunavir or a physiologically functional derivative thereof and
(d) ritonavir or a physiologically functional derivative thereof, wherein the ritonavir is in the form of its crystalline form II.

In one aspect, the present invention further provides a monolithic tablet comprising:
(a) tenofovir or a physiologically functional derivative thereof,
(b) emtricitabine or a physiologically functional derivative thereof,
(c) darunavir or a physiologically functional derivative thereof and
(d) ritonavir or a physiologically functional derivative thereof, wherein the ritonavir is in the form of its crystalline form II and/or wherein the amount of ritonavir is less than or equal to about 70 mg.

In one aspect, the present invention further provides a monolithic tablet comprising:
(a) about 300 mg of tenofovir disoproxil fumarate,
(b) about 200 mg of emtricitabine,
(c) about 800 mg of darunavir and
(d) less than or equal to about 70 mg of ritonavir.

In one aspect, the present invention further provides a monolithic tablet comprising:
(a) about 300 mg of tenofovir disoproxil fumarate,
(b) about 200 mg of emtricitabine,
(c) about 800 mg of darunavir and
(d) less than or equal to about 70 mg of ritonavir, wherein the ritonavir is in the form of its crystalline form II.

In one aspect, the present invention further provides a monolithic tablet in a unit dosage form comprising:
(a) about 300 mg of tenofovir disoproxil fumarate,
(b) about 200 mg of emtricitabine,
(c) about 800 mg of darunavir and
(d) ritonavir or a physiologically functional derivative thereof, wherein the amount of ritonavir is less than about 100 mg and wherein the ritonavir is in the form of a ritonavir pre-mix.

In one aspect, the present invention further provides a monolithic tablet comprising:
(a) darunavir or a physiologically functional derivative thereof (preferably darunavir hydrate or darunavir ethanolate); wherein the amount of darunavir is 800 mg and
(b) ritonavir or a physiologically functional derivative thereof; wherein the amount of ritonavir is less than or equal to about 70 mg.

In one aspect, the present invention further provides a monolithic tablet comprising:
(a) darunavir or a physiologically functional derivative thereof (preferably darunavir hydrate or darunavir ethanolate); wherein the amount of darunavir is 800 mg and
(b) ritonavir or a physiologically functional derivative thereof; wherein the amount of ritonavir is less than or equal to about 70 mg and wherein the ritonavir is in the form of ritonavir crystalline form II.

The present invention further provides the above unit dose formulation or the above monolithic tablet for use as a medicament.

The present invention further provides the above unit dose formulation or the above monolithic tablet for use in the treatment of HIV-1 infection.

The present invention further provides a method for treating HIV-1 infection comprising administration of a pharmaceutically effective amount of the above unit dose formulation or of the above monolithic tablet.

### DETAILED DESCRIPTION OF THE INVENTION

Unless stated otherwise, the following terms and phrases as used herein are intended to have the following meanings:

The compounds or their combinations of the invention may be referred to as “active ingredients” or “pharmaceutically active agents.”

The term “physiologically functional derivative” includes any: pharmaceutically acceptable salts, pharmaceutically acceptable enantiomers, pharmaceutically acceptable solid state form (crystalline, semi-crystalline or amorphous), pharmaceutically acceptable polymorphs, pharmaceutically acceptable solvates, pharmaceutically acceptable metabolites or pharmaceutically acceptable prodrugs thereof (for example, wherein the prodrug is an ester), or a pharmaceutically acceptable salt of the enantiomer, solid state form, polymorph, solvate, metabolite or prodrug (e.g. an ester prodrug).

The terms “Emtricitabine”, “Tenofovir”, “Darunavir” and “Ritonavir” are mentioned throughout in a broad sense to include not only emtricitabine, tenofovir, darunavir and ritonavir, per se, but also their physiologically functional derivatives. Preferably in any embodiment or any aspect of the present invention, the tenofovir is in the form of its prodrug, tenofovir disoproxil. More preferably the tenofovir disoproxil is in the form of its fumarate salt, i.e. tenofovir disoproxil fumarate. Preferably in any embodiment or any aspect of the present invention, the darunavir is in the form of darunavir or its physiologically functional derivatives, e.g. darunavir can be darunavir ethanolate, hydrate, or any other crystalline form as well as amorphous darunavir.

Preferably in any embodiment or any aspect of the present invention, the ritonavir is in the form of ritonavir or its physiologically functional derivatives. More preferably the ritonavir is in the form of its crystalline form as well as amorphous ritonavir, i.e., crystalline form can be for example, Form I or Form II, substantially as described in EP1097148. Characteristic peaks in the powder X-ray diffraction pattern of Ritonavir Form I can be found at 3.33°±0.1°, 6.76°±0.1°, 8.33°±0.1°, 14.61°±0.1°, 16.33°±0.1°, 16.76°±0.1°, 17.03°±0.1°, 18.02°±0.1°, 18.62°±0.1°, 19.47°±0.1°, 19.86°±0.1°, 20.25°±0.1°, 21.46°±0.1°, 23.46°±0.1° and 24.36°±0.1° (two theta). Characteristic peaks in the powder X-ray diffraction pattern of Ritonavir Form II can be found at 8.67°±0.1°, 9.88°±0.1°, 16.11°±0.1°, 16.70°±0.1°, 17.36°±0.1°, 17.78°±0.1°, 18.40°±0.1°, 18.93°±0.1°, 20.07°±0.1°, 20.65°±0.1°, 21.71°±0.1° and 25.38°±0.1° (two theta).

A ritonavir hot melt-extrusion process comprises the steps of preparing a homogeneous melt of ritonavir or a combination of ritonavir and another therapeutic agent(s), water-soluble polymer and surfactant, and then cooling the melt until it solidifies. “Melting” means a transition into a liquid or rubbery state in which it is possible for one component to get embedded homogeneously in the other. Typically, one component will melt and the active ingredient(s) will dissolve in the melt thus forming a solution. Melting usually involves heating above the softening point of the water-soluble polymer. The preparation of the melt can take place in a variety of ways. The mixing of the components can take place before, during or after the formation of the melt. For example, the components can be mixed first and then melted, or be simultaneously mixed and melted. Usually, the melt is homogenized in order to disperse the active ingredients efficiently. Also, it may be convenient first to melt the water-soluble polymer and then to mix in and homogenize the active ingredients.

Typically, the hot melt extrusion process requires the use of a large ratio of polymer to drug in order to form the melt. The process results in the production of mixture containing amorphous ritonavir in the polymeric mass. Due to the high polymer content required, the preparation of ritonavir compositions by hot melt extrusion is not preferred when formulating unit dosage forms containing more than one drug due to limitations on the size of the dosage form for administration by, e.g. swallowing.

Preferably, the ritonavir in the unit dosage form of the present invention can be prepared by a process that avoids the use of a hot melt extrusion process. For example, the ritonavir, which may preferably be crystalline Form I or Form II as described above, can be prepared by wet or dry granulation or by blending, optionally with excipients and/or one or more of the other active agents in the dosage form.

Unless otherwise indicated, a reference to weight % of the dosage form means the weight % relative to the weight of the dosage form excluding, in the case of a tablet, any finishing/cosmetic coating, and in the case of a capsule, the weight of the dosage form refers to the total weight of the capsule contents, i.e. excluding the capsule shell. In the case of a tablet, the finishing/cosmetic coating can add, e.g. about 2% to about 5%, preferably about 2 to about 4% and particularly about 3% by weight to the total weight of the tablet.

The term “chemical stability” means that the active ingredients in the combination are substantially stable to chemical degradation. Preferably, they are sufficiently stable in physical combination to permit commercially useful shelf life of the combination product. Typically, “chemically stable” means that a first component of the mixture does not act to degrade a second component when the two are brought into physical combination to form a pharmaceutical dosage form. Preferably in any embodiment of any aspect of the present invention, the term “chemically stable” refers to a formulation in which, when the formulation is stored at 40° C. and at 75% relative humidity for 1 month to 6 months, the amounts of each of darunavir and ritonavir; and/or the amount of each of tenofovir and emtricitabine, do not significantly diminish compared with the amounts of each of darunavir and ritonavir; and/or the amount of each of tenofovir and emtricitabine, in the formulation prior to storage.

In particular, a formulation according to any aspect or embodiment of the present invention may be considered to be chemically stable if at least about 97%, preferably at least about 98%, more preferably at least about 99%, most preferably at least about 99.5%, and especially at least about 99.9% of each of the darunavir and ritonavir content, and/or each of tenofovir and emtricitabine content in the formulation immediately before storage is retained after storage at 40° C. and at 75% relative humidity for 1 month to 6 months. Preferably, in accordance with any aspect or embodiment of the present invention, the term “chemically stable” refers to a formulation in which from about 90% to about 100%, about 95% to about 100%, about 98% to about 100%, about 99% to about 100%, 99.5% to about 100% or 99.9% to about 100% of each of the darunavir and ritonavir content and/or each of tenofovir and emtricitabine content in the formulation immediately before storage is retained following storage of the formulation at 40° C. and at 75% relative humidity for 1 month to 6 months. More preferably, about 99.95% to about 100% is retained. In a particularly preferred embodiment of the present invention, the term “chemically stable” refers to a formulation in which at least about 99.95% of each of the darunavir and ritonavir content and/or each of tenofovir and emtricitabine content in the formulation immediately before storage is retained after storage at 40° C. and at 75% relative humidity for 1 month to 6 months. In especially preferred embodiments of the present invention, the term “chemically stable” refers to a formulation in which there is no detectable change in the darunavir and ritonavir content and/or each of tenofovir and emtricitabine content, in the formulation immediately before storage and after storage at 40° C. and at 75% relative humidity for 1 month to 6 months.

Any change in the darunavir and ritonavir contents, and/or tenofovir and emtricitabine contents of the formulations can be measured by standard analytical techniques well known to the skilled person. HPLC is a preferred method for this purpose. For example, an HPLC assay using standard solutions may be employed, an example of which is set out below.

Reference to weight % or % by weight of pharmaceutically acceptable salts of emtricitabine, tenofovir, darunavir and ritonavir refer to the amount relative to the free base form. Where the emtricitabine, tenofovir, darunavir and/or ritonavir is in the form of a prodrug, the weight % refers to the amount relative to the form of the prodrug. Thus, for example, reference to weight % of tenofovir disoproxil fumarate is the amount relative to tenofovir disoproxil.

The term “bioavailability” means the rate and extent to which the active ingredient or active moiety is absorbed from a drug product and becomes available at the site of action. Enhancement of the bioavailability of a pharmaceutically active agent can provide a more efficient and effective treatment for patients because, for a given dose, more of the pharmaceutically active agent will be available at the targeted tissue sites.

The bioavailability of a pharmaceutical composition can be determined, for example, by any pharmacokinetic parameter known to the person skilled in the art. Examples of such parameters include: t_{1/2} (half-life), Cₘᵢₙ (minimal plasma concentration), C trough concentration, Cₘₐₓ (maximal plasma concentration), AUC (area under the curve), Tₘₐₓ (time to maximal concentration), and Cₛₛ (steady state concentration).

The assessment of same/comparable pharmacokinetic bioavailability can be based on 90% confidence intervals for the ratio of the population geometric means (test/reference) for the parameters under consideration. This method can be equivalent to two one-sided tests with the null hypothesis of bioinequivalence at the 5% significance level.

The pharmacokinetic parameters under consideration can be analyzed using ANOVA. The data can be transformed prior to analysis using a logarithmic transformation. A confidence interval for the difference between formulations on the log-transformed scale can be obtained from the ANOVA model. This confidence interval can then back-transformed to obtain the desired confidence interval for the ratio on the original scale.

Ritonavir, may be used as ‘pharmacokinetic enhancer’ (booster) to increase the blood levels of darunavir. Accordingly, the bioavailability of darunavir may be compared to the bioavailability of the commercial darunavir when administered with the commercial ritonavir (Norvir®) at recommended effective doses as a pharmacokinetic enhancer.

The references hereinafter to a pharmaceutical formulation refer unless otherwise stated to a pharmaceutical formulation containing the combination or also their physiologically functional derivatives.

The unit dosage form of the 4 APIs enable patients greater freedom from multiple dosage medication regimens and ease the needed diligence required in remembering and complying with complex daily dosing times and schedules. By combining tenofovir disoproxil, emtricitabine, darunavir and ritonavir into a single dosage form, the desired daily regimen may be presented in a single dose per day. The pharmaceutical formulations of co-formulated tenofovir, emtricitabine, darunavir, and ritonavir may be administered as a single dose form, once per day.

Ideally, the pharmaceutical formulation should be administered to achieve peak plasma concentrations of each of the compounds/active pharmaceutical ingredients when administered in a separated unit doses containing each one of the active pharmaceutical ingredients separately, while avoiding compromising the stability and the size of the pharmaceutical formulation. For example, emtricitabine, tenofovir and darunavir and ritonavir may be formulated to produce a composition in which the peak plasma concentrations of each of the active agents in the formulation are similar or the same as (i.e. “bioequivalent to”) the peak plasma concentrations achieved by separate administration of each active, when administered to an individual. In the case of ritonavir, the applicant has found that ritonavir-mediated pharmacokinetic enhancement of darunavir may still be achieved even if the peak plasma concentrations of the ritonavir in the formulations do not reach those of the commercially available 100 mg ritonavir tablet composition (Norvir®) (i.e. effective pharmacokinetic enhancement of darunavir by ritonavir in the compositions according to any aspect of the present invention does not require bioequivalence with the commercially available 100 mg Norvir® tablet). It was previously thought that while doses as low as 50 mg ritonavir may be sufficient to provide the same pharmacokinetic-boosting effect as 100 mg ritonavair, ritonavir had to be incorporated using an amorphous ritonavir in a hot melt extrusion manufacturing process, with a high amount of polymer. (Estévez, J., et al., J Antimicrob Chemother 2012, 67, 2013-2019; Hill, A., et al., AIDS 2009, 23, 2237-2245; Hill, A., et al., J Acquir Immune Defic Syndr, 2011, Vol. 58, No. 5; (NDA 22-417—chemistry review, http://www.accessdata.fda.gov/drugsatfda_docs/nda/2010/022417s000_ChemR.pdf)). Thus, the success of a low dosage of ritonavir in the absence of a hot melt extrusion manufacturing process was unexpected.

Thus, in preferred embodiments of the present invention the ritonavir in the formulations containing emtricitabine, tenofovir, darunavir and ritonavir, or the monolithic tablet comprising ritonavir and darunavir is not bioequivalent to the commercially available 100 mg ritonavir tablet composition (Norvir®). For example, the ritonavir can be present in any of the formulations of the present invention in an amount of less than about 100 mg, less than or equal to about 70 mg, less than or equal to about 60 mg, less than or equal to about 50 mg, e.g. the ritonavir can be present in any of the formulations of the present invention in an amount of between about 50 mg to about 60 mg, to about 70 mg, to about 80 mg, to about 100 mg, between about 60 mg to about 70 mg, to about 80 mg, to about 100 mg, between about 80 mg, to about 100 mg, preferably, the ritonavir can be present in any of the formulations of the present invention in an amount of about 70 mg.

“Non-bioequivalent” ritonavir refers to a formulation in which the ritonavir exhibits statistically significant difference in the bioavailability compared to the bioavailability of the reference product, i.e. the commercially available Norvir® tablet as marketed by Abbott Laboratories and as described above. Bioavailability and hence bioequivalence/non-bioequivalence can be determined by reference to pharmacokinetic parameters such as AUC₀₋ₜ, AUC_{0-∞} and Cₘₐₓ in accordance with the regulatory guidelines—for example US Food and Drug Administration (FDA) “Guidance for Industry”—Bioavailability and Bioequivalence Studies for Orally Administered Drug Products—General Considerations, March 2003, Revision 1; http://www.fda.gov/downloads/Drugs/ . . . /Guidances/ucm070124.pdf) or European Medicines Agency, Committee for Medicinal Products for Human Use: Guideline on the Investigation of Bioequivalence, 20 Jan. 2010, Document ref: CPMP/EWP/QWP/1401/98 Rev. 1/Corr**; http://www.ema.europa.eu/docs/en_GB/document_library/Scientific_guideline/2010/01/W C500070039.pdf). Accordingly, regulatory guidelines specify that bioequivalence requires pharmacokinetic parameters (particularly AUC₀₋ₜ and Cₘₐₓ) in which 90% confidence interval (CI) values of the ratio of the test and the reference product are within the range of at least 80.00% and not more than 125.00%. Hence, non-bioequivalence refers to pharmacokinetic parameters (particularly AUC₀₋ₜ and Cₘₐₓ) in which 90% confidence interval values of the ratio of the test and the reference product are outside of this range. However, for the present invention, non-bioequivalence for ritonavir preferably refers to pharmacokinetic parameters (preferably one or both of AUC₀₋ₜ and Cₘₐₓ) in which 90% confidence interval values of the ratio of the test (i.e. a formulation according to the present invention) and the reference product [i.e. commercially available 100 mg ritonavir (Norvir®)] is less than 80.00%. Preferably in any embodiment of the present invention, the ritonavir in the formulation is non-bioequivalent to the commercially available Norvir® tablet, particularly wherein the 90% confidence interval for the ratio of the ritonavir and the corresponding commercially available 100 mg ritonavir (Norvir®) is from about 30 up to but not including 80.00%, from about 35 up to but not including 80.00%, from about 40 up to but not including 80.00%, from about 45 up to but not including 80.00%, from about 50 up to but not including 80.00%, from about 55 up to but not including 80.00%, from about 60 up to but not including 80.00%, from about 65 up to but not including 80.00%, from about 70 up to but not including 80.00%, or from about 75% up to but not including 80.00%. Preferably, in any embodiment of the present invention, the ritonavir in the formulation is non-bioequivalent to the commercially available Norvir® tablet, particularly wherein the 90% confidence interval for the ratio of the ritonavir and the corresponding commercially available 100 mg ritonavir (Norvir®) is from about 30: to about 35, 40, 45, 50, 55, 60, 65, 70, or 75%; or from about 35: to about 40, 45, 50, 55, 60, 65, 70, or 75%; or from about 40: to about 45, 50, 55, 60, 65, 70, or 75%; or from about 45: to about 50, 55, 60, 65, 70 or 75%; or from about 50: to about 55, 60, 65, 70 or 75%; or from about 55: to about 60, 65, 70, or 75%; or from about 60: to about 65, 70, or 75%; or from about 70 to about 75%.

As used herein, unless otherwise indicated, “rate of release” or “release rate” of a drug refers to the amount or percentage (preferably percentage) of drug released from a dosage form per unit time, e.g., a weight % of drug released (relative to the total weight % of that drug in the dosage form) within a particular time (wt %/x minutes). Drug release rates for dosage forms are typically measured in vitro as an amount or percentage (preferably percentage) of drug released from the dosage form, after a particular time measured under appropriate conditions and in a suitable fluid. As referred to herein, unless indicated otherwise, drug release rate is determined using 1800 ml of 2% Brij-35 (30% solution in water) in 0.05M phosphate buffer, having a pH of 3.0, using paddles apparatus (USP apparatus II) at 75 rpm, at a temperature of 37° C.±0.5° C.

The pharmaceutical formulation may be formulated in a unit dosage formulation comprising an amount of each compound/active pharmaceutical ingredient that is suitable for a daily dose to ensure the desired therapeutic effect.

The present invention provides a pharmaceutical formulation in a unit dosage form comprising:

tenofovir or a physiologically functional derivative thereof,

emtricitabine or a physiologically functional derivative thereof,

darunavir or a physiologically functional derivative thereof; and

ritonavir or a physiologically functional derivative thereof; wherein the amount of ritonavir is less than about 100 mg.

The above pharmaceutical formulation comprising tenofovir disoproxil or a physiologically functional derivative thereof, emtricitabine or a physiologically functional derivative thereof, darunavir or a physiologically functional derivative thereof and ritonavir or a physiologically functional derivative thereof can be chemically stable.

The total weight of the pharmaceutical formulation of the present invention, comprising the above 4 APIs, can be less than or equal to about 2.800 g, less than or equal to about 2.600 g, less than or equal to about 2.500 g, less than or equal to about 2.400 g, less than or equal to about 2.300 g, less than or equal to about 2.200 g, less than or equal to about 1.900 g or less than or equal to about 1.800 g, or less than or equal to about 1.700 g, or less than or equal to about 1.600 g, or less than or equal to about 1.500 g. In a specific embodiment of the present invention the total dosage form weight is between 1.500 g to about 1.600 g, to about 1.700 g, to about 1.800 g, to about 1.900 g, to about 2.000 g, to about 2.200 g, to about 2.300 g, to about 2.400 g, to about 2.500 g, to about 2.600 g, to about 2.800 g, or between 1.600 g, to about 1.700 g, to about 1.800 g, to about 1.900 g, to about 2.000 g, to about 2.200 g, to about 2.300 g, to about 2.400 g, to about 2.500 g, to about 2.600 g, to about 2.800 g, or between 1.700 g, to about 1.800 g, to about 1.900 g, to about 2.000 g, to about 2.200 g, to about 2.300 g, to about 2.400 g, to about 2.500 g, to about 2.600 g, to about 2.800 g, or between 1.800 g, to about 1.900 g, to about 2.000 g, to about 2.200 g, to about 2.300 g, to about 2.400 g, to about 2.500 g, to about 2.600 g, to about 2.800 g, or between 1.900 g, to about 2.000 g, to about 2.200 g, to about 2.300 g, to about 2.400 g, to about 2.500 g, to about 2.600 g, to about 2.800 g, or between 2.000 g to about 2.200 g, to about 2.300 g, to about 2.400 g, to about 2.500 g, to about 2.600 g, to about 2.800 g, or between about 2.200 g, to about 2.300 g, to about 2.400 g, to about 2.500 g, to about 2.600 g, to about 2.800 g, or between about 2.300 g, to about 2.400 g, to about 2.500 g, to about 2.600 g, to about 2.800 g, or between about 2.400 g, to about 2.500 g, to about 2.600 g, to about 2.800 g, or between about 2.500 g, to about 2.600 g, to about 2.800 g, or between about 2.600 g to about 2.800 g.

By way of example, the pharmaceutical formulation of the present invention comprises about 150 mg to 350 mg of tenofovir disoproxil fumarate, about 100 mg to 300 mg of emtricitabine, about 75 mg to 800 mg of darunavir and about 100 mg of ritonavir. The amount of darunavir can be 75 mg, 150 mg, 300 mg, 400 mg 600 mg, or 800 mg (corresponding to the doses of commercially available of Prezista®). The said darunavir can be darunavir ethanolate, hydrate, or any other crystalline form as well as darunavir amorphous. The amount of ritonavir can be less than about 100 mg, less than or equal to about 70 mg, less than or equal to about 60 mg, less than or equal to about 50 mg, e.g. the amount of ritonavir can be between about 50 mg to about 60 mg, to about 70 mg, to about 80 mg, to about 100 mg, between about 60 mg to about 70 mg, to about 80 mg, to about 100 mg, between about 80 mg, to about 100 mg, preferably, the amount of ritonavir can be about 70 mg.

The said ritonavir can be in a crystalline form as well as amorphous ritonavir, for example, the said ritonavir can be in a crystalline form and/or amorphous ritonavir, i.e., the crystalline form can be for example, Form I or Form II, preferably, ritonavir Form II.

Preferably, the pharmaceutical formulation of the present invention comprises about 300 mg of tenofovir disoproxil fumarate, about 200 mg of emtricitabine, about 800 mg of darunavir and less than or equal to about 70 mg of ritonavir.

In certain embodiments, the pharmaceutical formulation of the present invention comprises from about 20% to about 85% by weight of total weight of all four pharmaceutically active ingredients. The pharmaceutical formulation of the present invention comprises from about 20% to about 25%, to about 30%, to about 35%, to about 40%, to about 45%, to about 50%, to about 55%, to about 60%, to about 65%, to about 70%, to about 75%, to about 80%, or to about 85% by weight of total weight of all 4 pharmaceutically active ingredients, or from about 25% to about 30%, to about 35%, to about 40%, to about 45%, to about 50%, to about 55%, to about 60%, to about 65%, to about 70%, to about 75%, to about 80%, or to about 85%, or from about 30% to about 35%, to about 40%, to about 45%, to about 50%, to about 55%, to about 60%, to about 65%, to about 70%, to about 75%, to about 80%, or to about 85%, or from about 40% to about 45%, to about 50%, to about 55%, to about 60%, to about 65%, to about 70%, to about 75%, to about 80%, or to about 85%, or from about 45% to about 50%, to about 55%, to about 60%, to about 65%, to about 70%, to about 75%, to about 80%, or to about 85%, or from about 50% to about 55%, to about 60%, to about 65%, to about 70%, to about 75%, to about 80%, or to about 85% or from about 55%, to about 60%, to about 65%, to about 70%, to about 75%, to about 80%, or to about 85%, or from about 60% to about 65%, to about 70%, to about 75%, to about 80%, or to about 85%, or from about 65% to about 70%, to about 75%, to about 80%, or to about 85%, or from about 70% to about 75%, to about 80%, or to about 85%, or from about 75%, to about 80%, or to about 85%, or from about 80% to about 85% by weight of all four pharmaceutically active ingredients.

The pharmaceutical formulation of the present invention can be suitable for oral administration. Oral dosage forms for the purpose of the present invention include capsules, tablets, pellets, granules, powders and pharmaceutical formulations thereof. For example, the pharmaceutical composition can be formulated in the form of coated or uncoated, effervescent, soluble, orodispersible, enteric or modified-release tablets; sugar-coated tablets; hard capsules; soft capsules; granules; pills; pastilles. Preferably, the oral dosage form is a tablet. The tablet can be chemically stable.

The tablet of any embodiment of the present invention may be divisible into one or more subunits. For example, the tablet may be provided with one or more score lines (e.g. indentations or grooves) enabling it to be divided into a plurality of parts. For example, a tablet of any embodiment of the present invention may be provided with score lines to enable the tablet to be divided into two, three or four parts. Thus, for example, according to an embodiment of the present invention, the tablet may be provided with a score line across the tablet, enabling it to be divided into two (preferably substantially equal) parts—e.g. for a round tablet, a score line may be provided across a diameter of the tablet, enabling the tablet to be divided (broken) into two substantially equal halves. A tablet according to any embodiment of the present invention may be provided with two score lines enabling it to be divided into three (preferably substantially equal) portions. Alternatively, in the case of a round tablet, the tablet may be provided with three score lines extending radially from the centre, enabling the tablet to be divided into three parts. A tablet according to any embodiment of the invention may be provided with three score lines, in order to enable the tablet to be divided into four (preferably substantially equal) parts. Alternatively, in the case of a round tablet, the tablet may be provided with two perpendicular score lines across the diameter of the tablet, thus enabling the tablet to be divided into four parts. In any embodiment of the present invention, the score line(s) may provided on one face of the tablet, and optionally additionally on the sides of the tablet to further facilitate division of the tablet. Alternatively, the score line(s) may be provided on both faces of the tablet and optionally additionally on the sides of the tablet. The score lines are preferably formed by a continuous indentation (groove) across a surface of the tablet.

Score lines may be provided by any known method. Preferably, score lines are provided during the tablet compression step. For example, the die used for the compression can be shaped so as to provide a score line during tablet compression.

The tablet can have a weight as mentioned above for the total weight of the pharmaceutical formulation of the present invention, comprising the above 4 APIs.

The largest maximum diameter of the tablet of the present invention, comprising the 4 APIs, can be about 27 mm or less and the depth can be less than about 12.5 mm; preferably, the largest maximum diameter can be between about 26 mm to about 27 mm and the depth can be between about 12.5 mm to about 10 mm; more preferably, the largest maximum diameter can be between about 25 mm to about 26 mm and the depth can be between about 12 mm to about 11 mm, most preferably, the largest maximum diameter can be about 25 mm and the depth can be about 11 mm.

The hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU as measured by Electronic D-64291 Darmstadt 100-240V.

By way of example, the tablet of the present invention comprises about 150 mg to 350 mg of tenofovir disoproxil fumarate, about 100 mg to 300 mg of emtricitabine, about 75 mg to 800 mg of darunavir and about 100 mg, or <100 mg, or preferably <70 mg, of ritonavir. The amount of darunavir can be 75 mg, 150 mg, 300 mg, 400 mg 600 mg, or 800 mg (corresponding to the doses of commercially available of Prezista®).

By way of example, the total tablet weight of the tablet of the present invention comprising:
a) about 300 mg of tenofovir disoproxil fumarate,
b) about 200 mg of emtricitabine,
c) about 800 mg of darunavir; and
d) ritonavir or a physiologically functional derivative thereof, wherein the amount of ritonavir is less than about 100 mg, can be less than or equal to about 2.800 g, less than or equal to about 2.600 g, less than or equal to about 2.500 g, less than or equal to about 2.400 g, less than or equal to about 2.300 g, less than or equal to about 2.200 g, less than or equal to about 1.900 g or less than or equal to about 1.800 g, or less than or equal to about 1.700 g, or less than or equal to about 1.600 g, or less than or equal to about 1.500 g.

In a specific embodiment of the present invention, the total tablet weight of the tablet of the present invention comprising:
a) about 300 mg of tenofovir disoproxil fumarate,
b) about 200 mg of emtricitabine,
c) about 800 mg of darunavir; and
d) ritonavir or a physiologically functional derivative thereof, wherein the amount of ritonavir is less than about 100 mg, is between 1.500 g to about 1.600 g, to about 1.700 g, to about 1.800 g, to about 1.900 g, to about 2.000 g, to about 2.200 g, to about 2.300 g, to about 2.400 g, to about 2.500 g, to about 2.600 g, or to about 2.800 g.

The said darunavir can be darunavir ethanolate, hydrate, or any other crystalline form as well as darunavir amorphous. The amount of ritonavir can be less than about 100 mg, less than or equal to about 70 mg, less than or equal to about 60 mg, less than or equal to about 50 mg, e.g. the amount of ritonavir can be between about 50 mg to about 60 mg, to about 70 mg, to about 80 mg, to about 100 mg, between about 60 mg to about 70 mg, to about 80 mg, to about 100 mg, between about 80 mg, to about 100 mg, preferably, the amount of ritonavir can be about 70 mg.

The said ritonavir can be in a crystalline form as well as amorphous ritonavir, for example, the said ritonavir can be in a crystalline form and/or amorphous ritonavir, i.e., crystalline form can be for example, Form I or Form II, preferably, ritonavir Form II.

Preferably, the tablet of the present invention comprises about 300 mg of tenofovir disoproxil fumarate, about 200 mg of emtricitabine, about 800 mg of darunavir and less than or equal to about 70 mg of ritonavir.

The present invention further provides a pharmaceutical formulation in a unit dosage form comprising:
(a) tenofovir or a physiologically functional derivative thereof,
(b) emtricitabine or a physiologically functional derivative thereof,
(c) darunavir or a physiologically functional derivative thereof and
(d) ritonavir or a physiologically functional derivative thereof, wherein the amount of ritonavir is less than about 100 mg, and wherein the ritonavir is in the form of a pre-mix, and wherein:
   at least 75% to 80% of tenofovir is released from the formulation within 10 minutes, or
   at least 70% to 80% of emtricitabine is released from the formulation within 10 minutes, or
   at least 25% to 30% of darunavir is released from the formulation within 10 minutes, or 60% to 65% of darunavir is released from the formulation within 30 minutes; or
   at least 45% to 55% of ritonavir is released from the formulation within 10 minutes, or 65% to 75% of ritonavir is released from the formulation within 30 minutes.

Preferably, the pharmaceutical formulation may provide a release rate of tenofovir, emtricitabine, darunavir and ritonavir as specified above.

The present invention additionally provides a pharmaceutical formulation in a unit dosage form comprising:
(a) tenofovir or a physiologically functional derivative thereof,
(b) emtricitabine or a physiologically functional derivative thereof,
(c) darunavir or a physiologically functional derivative thereof and
(d) ritonavir or a physiologically functional derivative thereof, wherein the amount of ritonavir is less than about 100 mg, and wherein the ritonavir is in the form of crystalline form II, and wherein:
   at least 75% to 80% of tenofovir is released from the formulation within 10 minutes, or
   at least 70% to 80% of emtricitabine is released from the formulation within 10 minutes, or
   at least 25% to 30% of darunavir is released from the formulation within 10 minutes, or 60% to 65% of darunavir is released from the formulation within 30 minutes; or
   at least 40% to 45% of ritonavir is released from the formulation within about 10 minutes, or 70% to 75% of ritonavir is released from the formulation within 30 minutes.

In certain embodiments, the tablet of the present invention comprises from about 20% to about 70% by weight of total weight of the pharmaceutically active ingredients. The tablet of the present invention comprises from about 20% to about 85% by weight of total weight of all 4 pharmaceutically active ingredients. The pharmaceutical formulation of the present invention comprises from about 20% to about 25%, to about 30%, to about 35%, to about 40%, to about 45%, to about 50%, to about 55%, to about 60%, to about 65%, to about 70%, to about 75%, to about 80%, or to about 85% by weight of total weight of all 4 pharmaceutically active ingredients, or from about 25% to about 30%, to about 35%, to about 40%, to about 45%, to about 50%, to about 55%, to about 60%, to about 65%, to about 70%, to about 75%, to about 80%, or to about 85%, or from about 30% to about 35%, to about 40%, to about 45%, to about 50%, to about 55%, to about 60%, to about 65%, to about 70%, to about 75%, to about 80%, or to about 85%, or from about 40% to about 45%, to about 50%, to about 55%, to about 60%, to about 65%, to about 70%, to about 75%, to about 80%, or to about 85%, or from about 45% to about 50%, to about 55%, to about 60%, to about 65%, to about 70%, to about 75%, to about 80%, or to about 85%, or from about 50% to about 55%, to about 60%, to about 65%, to about 70%, to about 75%, to about 80%, or to about 85% or from about 55%, to about 60%, to about 65%, to about 70%, to about 75%, to about 80%, or to about 85%, or from about 60% to about 65%, to about 70%, to about 75%, to about 80%, or to about 85%, or from about 65% to about 70%, to about 75%, to about 80%, or to about 85%, or from about 70% to about 75%, to about 80%, or to about 85%, or from about 75%, to about 80%, or to about 85%, or from about 80% to about 85% by weight of all 4 pharmaceutically active ingredients.

According to some embodiments, the present invention provides tablets which optionally can be divided into two or more discrete segments, for example, by dividing grooves. Said dividing grooves facilitate breaking the dosage into the corresponding fragments and therefore provide an easy division into partial doses which contain approximately equal proportions of the active substances. The tablets of the present invention may, for example have one to three, preferably two, laterally extending grooves on the top and bottom surfaces and a small laterally opening groove on the sides, to facilitate ease in breaking the tablet.

Pharmaceutical formulations in the form of a tablet according to the present invention can be provided for example as a monolithic tablet (single layer), bi-layer (two layers) or multi-layer tablet (three or more distinct layers), preferably the formulation can be a monolithic tablet.

A monolithic tablet according to the present invention can include the four compounds/active pharmaceutical ingredients mixed and compressed to a single layer tablet. The single layer can include darunavir with extra-granular ritonavir or darunavir with intra-granular ritonavir, (i.e., a granulate of darunavir-ritonavir) emtricitabine granules, and tenofovir granules, or granules comprising both emtricitabine and tenofovir.

Alternatively, the single layer can include a granulate of darunavir and granules comprising emtricitabine, tenofovir and ritonavir.

The granulate of darunavir-ritonavir can be prepared by wet granulation of darunavir. The wet granulation can include top spray process of at least one binder such as hypromellose dissolved in a suitable liquid (e.g., water) on darunavir, obtaining a wet granulate followed by drying the wet granulate and milling the dry granulate.

Darunavir can be mixed with at least one disintegrant such as Croscarmellose Sodium (Ac-Di-Sol®), prior to the top spray process. The obtained milled granulate of darunavir can then be mixed with ritonavir pre-mix or with ritonavir Form II, at least one filler such as microcrystalline cellulose, at least one disintegrant such as crospovidone, and at least one glidant such as silicon dioxide.

Then at least one lubricant such as sodium stearyl fumarate can be added to the obtained mixture to obtain the final blend of darunavir-ritonavir (i.e., darunavir with extra-granular ritonavir).

Ritonavir form II can be wet granulated prior to the mixing with the granulate of darunavir and the other excipients, in providing the darunavir with extra-granular ritonavir. For example, ritonavir form II can be wet granulated with at least one filler such as microcrystalline cellulose, followed by drying the wet granulate and milling the dry granulate.

Alternatively, the granulate of darunavir-ritonavir can be prepared by wet granulation, at least one binder such as hypromellose dissolved in a suitable liquid (e.g. water), by a top spray process on a mixture of darunavir and ritonavir pre-mix or on a mixture of darunavir and ritonavir Form II, followed by drying the wet granulate and milling the dry granulate. Darunavir and ritonavir can be mixed with at least one disintegrant such as Croscarmellose Sodium (Ac-Di-Sol®), prior to the top spray process. The obtained granulate of darunavir and ritonavir, can then be mixed with at least one filler such as microcrystalline cellulose, at least one disintegrant such as crospovidone, and at least one glidant such as silicon dioxide. Optionally, castor oil can be added to the obtained mixture. Then at least one lubricant such as sodium stearyl fumarate can be added to the obtained mixture to obtain the final blend of darunavir-ritonavir (i.e., darunavir with intra-granular ritonavir).

The disintegrant used prior to the top spray process, can be in an amount of about 2% to about 30% by weight of total weight of the tablet, or from about 5% to about 30%, or from about 5% to about 20%, or from about 5% to about 15%, or from about 5% to 10% by weight of total weight of the tablet. Preferably, the weight ratio of disintegrant (preferably croscarmellose sodium (Ac-Di-Sol®) to darunavir is from about 1:5 to about 1:25, preferably from about 1:8 to about 1:20, more preferably from about 1:10 to about 1:15.

The ritonavir pre-mix can be a co-precipitate of ritonavir with a pharmaceutically acceptable carrier, and optionally other pharmaceutically acceptable excipients. The pharmaceutically acceptable carrier can preferably be copovidone. Other pharmaceutically acceptable excipients in the coprecipitate may include a solubilizer (such as sorbitan laurate), and a glidant (such as colloidal silica). In a preferred embodiment of any aspect of the present invention, the ritonavir pre-mix can be a coprecipitate comprising ritonavir and copovidone. More preferably, the ritonavir pre-mix can be a coprecipitate comprising ritonavir, copovidone, sorbitan laurate and colloidal silica. The ritonavir in the pre-mix can be preferably in an amorphous form.

Preferably, the ritonavir is in the form of a pre-mix comprising a co-precipitate of ritonavir with a pharmaceutically acceptable carrier, and optionally other pharmaceutically acceptable excipients. Copovidone is a preferred pharmaceutically acceptable carrier. Other pharmaceutically acceptable excipients may include: a solubilizer (preferably sorbitan laurate), and/or a glidant (preferably colloidal silica). The inventors have surprisingly discovered that using ritonavir in the form of a pre-mix eliminates the need for a hot melt extrusion manufacturing process, while still allowing for doses of ritonavir that are less than those of the commercially available tablet.

A preferred pre-mix comprises ritonavir in the form of a co-precipitate comprising ritonavir, copovidone, sorbitan laurate and colloidal silica. Preferably, the co-precipitate of ritonavir and a carrier (preferably copovidone) contains a weight ratio of carrier:ritonavir of: about 1:2 to about 1:10, about 1:2 to about 1:7, about 1:3 to about 1:7, about 1:3 to about 1:6, or about 1:3 to about 1:5.

Preferably, the co-precipitate comprises the carrier, preferably copovidone, in an amount of about 5 to about 30 wt %, about 10 to about 28 wt %, about 12 to about 25 wt %, or about 15 to about 23 wt %. Preferably, the co-precipitate comprises ritonavir in an amount of about 50 to about 95 wt %, about 60 to about 85 wt %, about 70 to about 85 wt %, about 70 to about 80 wt %, about 72 to about 80 wt %, or about 75 to about 77 wt %.

Ritonavir pre-mix can be prepared by mixing ritonavir with at least one carrier such as copovidone, optionally also with at least one solubilizer such as sorbitan laurate and with at least one glidant such as silica, providing a mixture that is then combined with a solvent, such as ethanol to yield a second mixture. The solvent is then removed from the second mixture by evaporation techniques such as spray drying. The resulting mixture may be in the form of a powder, which may be subjected to a particle size reduction step (e.g., by milling, to D(0.1) about 9 μm, D(0.5) about 55 μm, D(0.9) about 176 μm).

Granules comprising both emtricitabine and tenofovir can be prepared by combining both APIs and at least one filler such as microcrystalline cellulose, at least one binder such as pregelatinized starch and at least one disintegrant such as croscarmellose sodium, wet granulating the mixture with water, drying the wet granulate and milling the dry granulate. The obtained granulate of emtricitabine and tenofovir can be mixed with the final blend of darunavir-ritonavir (i.e., darunavir with extra-granular ritonavir or darunavir with intra-granular ritonavir, as described above) and at least one filer such as microcrystalline cellulose, at least one disintegrant selected from: croscarmellose sodium (Ac-Di-Sol®) and/or crospovidone, and at least one glidant such as silicon dioxide. Then at least one lubricant such as sodium stearyl fumarate can be added to the obtained mixture to obtain to form a blend that can then be compressed into a tablet core. The tablet core can then be coated with a film coating material to produce a film coated tablet.

Alternatively, the granules comprising both emtricitabine and tenofovir can be prepared by combining both APIs and at least one filler selected from: microcrystalline cellulose, lactose and/or pregelatinized starch and at least one disintegrant such as croscarmellose sodium to obtain a powdery mixture that can then combined with water in the wet granulation process. The wet granulation process provides wet granules that can then be dried.

Alternatively emtricitabine and tenofovir can be granulated by the same method separately to produce separate emtricitabine and tenofovir granulates.

Alternatively, emtricitabine granulate can be prepared by combining emtricitabine with at least one filler selected from: microcrystalline cellulose, pregelatinized starch and at least one disintegrant such as croscarmellose sodium to obtain a powdery mixture that can then be granulated with water in the wet granulation process. The wet granulation process provides wet granules that can then be dried.

Tenofovir granulate can be prepared by combining Tenofovir with at least one filler selected from: microcrystalline cellulose and pregelatinized starch to obtain a powdery mixture that can then granulated with water in the wet granulation process. The wet granulation process provides wet granules that can then be dried.

Granules of emtricitabine, tenofovir and ritonavir can be prepared by combining emtricitabine, tenofovir and ritonavir form II with at least one filler such as microcrystalline cellulose, at least one binder such as pregelatinized starch and optionally at least one disintegrant such as croscarmellose sodium to obtain a powdery mixture that can then combined with water in the wet granulation process, followed by drying the wet granulate and milling the dry granulate.

A bi-layer tablet comprising emtricitabine, tenofovir, darunavir and ritonavir according to the present invention is composed of two distinct layers of the compounds/active pharmaceutical ingredients. For example, the first layer can have emtricitabine and tenofovir and the second can have darunavir and ritonavir. The first layer may further include at least one lubricant such as magnesium stearate to form the blend of the first layer. The blend of the second layer can include the above described granulate of darunavir-ritonavir (i.e., darunavir and ritonavir as extra-granular or darunavir and ritonavir as intra-granular). Both blends can then be compressed into one tablet core that has two distinct layers. The tablet core can then be coated with film coating material.

A multi-layer tablet according to the present invention is composed of three or more distinct layers of the compounds/active pharmaceutical ingredients.

Pharmaceutical formulations in the form of a tablet according to the present invention may be uncoated or may be coated by known techniques including microencapsulation to delay disintegration and absorption in the gastrointestinal tract and thereby provide a sustained action over a longer period. For example, a time delay material such as glyceryl monostearate or glyceryl distearate alone or with a wax may be employed.

The pharmaceutical formulations in the form of a tablet according to the present invention may be further coated with a coating layer. The coating layer may comprise, for example, a protective top coat which is disposed over the drug layer. According to some embodiments, the protective top coat layer may comprise a coating polymer and optionally one or more excipients such as, for example, a plasticiser, an anti-adherent or glidant, one or more pigments/opacifying agents, and combinations thereof.

The coating may be a water-soluble film coating that has no influence on the release of the active substance. The thickness of a soluble film coating may be from about 20 μm to about 100 μm.

Suitable film coating materials include, for example, cellulose derivatives, such as cellulose ethers, for example methylcellulose, hydroxypropylcellulose or hydroxypropylmethylcellulose; mixtures of polyvinyl pyrrolidone or of a copolymer of polyvinyl pyrrolidone and polyvinyl acetate with hydroxypropylmethylcellulose; mixtures of shellac with hydroxypropylmethylcellulose, polyvinyl acetate or copolymers thereof with polyvinyl pyrrolidone; or mixtures of water-soluble cellulose derivatives, such as hydroxypropylmethylcellulose, and water-insoluble ethylcellulose. These coating agents may, if desired, be used in admixture with other adjuncts, such as talc, wetting agents, for example polysorbates (for example for facilitating application), or pigments (for example, for marking purposes). Depending on the solubility of the constituents, these coatings can be applied in aqueous solution or in organic solution (for example, solutions of shellac or ethylcellulose in organic solvents). Mixtures of acrylates that are water-insoluble per se may also be used. For example, the copolymer of ethyl acrylate and methyl methacrylate may be used in an aqueous dispersion, with one or more water-soluble adjuncts, such as lactose, polyvinyl pyrrolidine, polyethylene glycol or hydroxypropylmethylcellulose.

Suitable plasticizers for the protective top coat include, for example, triacetin, diethyl phthalate, dibutyl sebacate, tributyl sebacate polyethylene glycol, and mixtures thereof. Polyethylene glycol is a preferred plasticizer.

Suitable anti-adherent or glidants for the protective top coat include, for example, talc, fumed silica, magnesium stearate, and mixtures thereof.

The tablet of the present invention can be prepared by a process comprising:

(a) blending a mixture comprising tenofovir disoproxil, emtricitabine, darunavir, ritonavir and optionally one or more pharmaceutically acceptable excipients;

(b) compressing the blend into tablet cores; and

(c) optionally, applying one or more coating layers over the tablet cores.

Preferably, the tablet obtained by this process is a monolithic tablet.

Alternatively, the tablet of the present invention can be prepared by a process comprising:

(a) providing two blend layers, wherein:
(i) the first layer comprises either a mixture of emtricitabine granulates and tenofovir granulates, or a granulate comprising a mixture of emtricitabine and tenofovir; and
(ii) the second layer comprises darunavir with extra-granular ritonavir or darunavir with intra-granular ritonavir, (i.e., a granulate of darunavir-ritonavir);

(b) compressing the two blend layers into a tablet; and

(c) optionally applying one or more coating layers over the tablet cores.

Preferably, the tablet obtained by this process is a bi-layer tablet. Blending can be accomplished using equipment such as tumble blender.

Compression can be accomplished using for example, the blend of active ingredients and excipients is passed through a roller apparatus for compaction. However, other means for compacting the API mixture, e.g., compaction into slugs (or “slugging”), may be used.

Alternatively, the active pharmaceutical ingredients blend of step (a) can be processed into other unitary dosage forms such as capsules, or the like.

Each of the individual active pharmaceutical ingredients in the mixture in step (a) can be processed prior to blending as described above.

Wet granulation can be accomplished using conventional equipment. For example, the powders were blended in a granulator and then granulated using water. The impeller and chopper speeds were kept constant in the blender at a low setting during the granulation and wet massing operations. After water addition, the impeller and chopper were stopped and the granulator bowl was opened to observe the granulation consistency and texture.

Wet milling can be accomplished using conventional equipment. For example, to facilitate a uniform drying process, each wet granulation can be deagglomerated with a mill fitted with a screen and an impeller.

Drying can be accomplished using conventional equipment. For example, milled wet granules can be dried using fluid-bed dryer.

Dry milling can be accomplished using conventional equipment. For example, all dried granules can be milled using a rotary sieve mill.

Top spray process can be accomplished using conventional equipment such as, For example, a fluid bed dryer equipped with either top spray or wurster coating devices.

Tablet compression can be accomplished using conventional equipment. For example, the final blends can be compressed using tabletting machine. Purity analysis can be accomplished using conventional equipment, for example HPLC.

The present invention provides a monolithic tablet comprising:

darunavir or a physiologically functional derivative thereof, and

ritonavir or a physiologically functional derivative thereof.

Preferably, the tablet can be chemically stable.

The total weight of the monolithic tablet comprising darunavir and ritonavir of the present invention can be less than or equal to about 1.300 g, less than or equal to about 1.200 g, less than or equal to about 1.100 g, less than or equal to about 1.000 g, less than or equal to about 0.900 g, less than or equal to about 0.700, less than or equal to 0.600 g, or less than or equal to about 0.550 g. In a specific embodiment of the present invention the tablet weight is between 0.550 g to about 0.600 g, to about 0.700 g, to about 0.900 g, to about 1.000 g, to about 1.100 g, to about 1.200 g, to about 1.300 g, or between 0.600 g to about 0.700 g, to about 0.900 g, to about 1.000 g, to about 1.100 g, to about 1.200 g, or to about 1.300 g, or between 0.700 g to about 0.900 g, to about 1.000 g, to about 1.100 g, to about 1.300 g, or between 0.900 g to about 1.000 g, to about 1.100 g, to about 1.200 g, or to about 1.300 g, or between 1.000 g to about 1.100 g, to about 1.200 g, to about 1.300 g, or between 1.100 g to about 1.200 g, or to about 1.300 g, or between 1.200 g to about 1.300 g.

The monolithic tablet comprising darunavir and ritonavir of the present invention provides a chemically stable formulation of the active agents, and moreover, is suitable for once daily administration in a single pill. Advantageously, the size and weight of the dosage form is far less than the combined size/weight of commercially available darunavir 800 mg tablet (Prezista®, having total weight of 1048 mg) and commercially available ritonavir 100 mg tablet (Norvir®, having a total weight of 800 mg).

The largest maximum diameter of the pharmaceutical formulation of the present invention, comprising darunavir and ritonavir, can be about 22 mm or less and the depth can be less than about 9 mm; preferably, the largest maximum diameter can be between about 20 mm to about 22 mm and the depth can be between about 6 mm to about 9 mm; more preferably, the largest maximum diameter can be between about 20 mm to about 21 mm and the depth can be between about 8 mm to about 7 mm, most preferably, the largest maximum diameter can be about 21 mm and the depth can be about 7 mm.

By way of example, the tablet of the present invention may comprise about 150 mg to 350 mg of tenofovir disoproxil fumarate, about 100 mg to 300 mg of emtricitabine, about 75 mg to 800 mg of darunavir and about 100 mg or less of ritonavir. The amount of darunavir can be 75 mg, 150 mg, 300 mg, 400 mg, 600 mg, or 800 mg (corresponding to the doses of commercially available of Prezista®). The said darunavir can be darunavir ethanolate, hydrate, or any other crystalline form as well as darunavir amorphous.

The amount of ritonavir can be less than about 100 mg, less than or equal to about 90 mg, less than or equal to about 85 mg, less than or equal to about 80 mg, less than or equal to about 75 mg, less than or equal to about 70 mg, less than or equal to about 60 mg, less than or equal to about 50 mg, e.g. the amount of ritonavir can be between about 50 mg to about 60 mg, to about 70 mg, to about 80 mg, to about 85 mg, to about 90 mg, to about 100 mg, between about 60 mg to about 70 mg, to about 80 mg, to about 85 mg, to about 90 mg, to about 100 mg, between about 80 mg to about 100 mg, between about 80 mg to about 90 mg, preferably, the amount of ritonavir can be about 70 mg or about 85 mg.

The said ritonavir can be in a crystalline form as well as amorphous ritonavir, for example, the said ritonavir can be in a crystalline form and/or amorphous ritonavir, i.e., crystalline form can be for example, Form I or Form II, preferably, ritonavir Form II.

Preferably, the tablet of the present invention comprises about 300 mg of tenofovir disoproxil fumarate, about 200 mg of emtricitabine, about 800 mg of darunavir and less than or equal to about 70 mg or less than or equal to about 85 mg of ritonavir.

In certain embodiments, the monolithic tablet comprising darunavir and ritonavir of the present invention comprises from about 20% to about 85% by weight of total weight of the two pharmaceutically active ingredients. The tablet of the present invention comprises from about 20% to about 25%, to about 30%, to about 35%, to about 40%, to about 45%, to about 50%, to about 55%, to about 60%, to about 65%, to about 70%, to about 75%, to about 80% or to about 85% by weight of total weight of the two pharmaceutically active ingredients, or from about 25% to about 30%, to about 35%, to about 40%, to about 45%, to about 50%, to about 55%, to about 60%, to about 65%, to about 70%, to about 75%, to about 80%, or to about 85%, or from about 30% to about 35%, to about 40%, to about 45%, to about 50%, to about 55%, to about 60%, to about 65%, to about 70%, to about 75%, to about 80%, or to about 85%, or from about 40% to about 45%, to about 50%, to about 55%, to about 60%, to about 65%, to about 70%, to about 75%, to about 80%, or to about 85%, or from about 45% to about 50%, to about 55%, to about 60%, to about 65%, or to about 70%, to about 75%, to about 80% or to about 85%, or from about 50% to about 55%, to about 60%, to about 65%, to about 70%, to about 75%, to about 80%, or to about 85%, or from about 55%, to about 60%, to about 65% or to about 70%, to about 75%, to about 80% or to about 85%, or from about 60% to about 65%, to about 70%, to about 75%, to about 80%, or to about 85%, or from about 65%, to about 70%, to about 75%, to about 80%, or to about 85%, or from about 70% to about 75%, to about 80%, or to about 85%, or form about 75% to about 80% or to about 85%, or from about 80% to about 85% by weight of the two pharmaceutically active ingredients.

The above monolithic tablet comprising darunavir and ritonavir can include the two compounds/active pharmaceutical ingredients mixed and compressed to a single layer tablet. The single layer can include darunavir and ritonavir as extra-granular or darunavir and ritonavir as intra-granular. To these can be added at least one lubricant such as sodium stearyl fumarate to form a blend that can then be compressed into a tablet core. The tablet core can then be coated with a film coating material to produce a film coated tablet.

Darunavir and ritonavir as extra-granular or intra-granular can be processed prior to blending as described above.

According to some embodiments, the present invention provides tablets which optionally can be divided into two or more discrete segments, for example, by dividing grooves. Said dividing grooves facilitate breaking the dosage into the corresponding fragments and therefore provide an easy division into partial doses which contain approximately equal proportions of the active substances. The tablets of the present invention may, for example have one to three, preferably two, laterally extending grooves on the top and bottom surfaces and a small laterally opening groove on the sides, to facilitate ease in breaking the tablet.

The pharmaceutical formulations of the present invention may further comprise one or more pharmaceutically acceptable carriers or excipients as described above and below.

Examples of pharmaceutical excipients are fillers, binders, disintegrants, surfactants, glidants and lubricants.

Suitable fillers (diluents) include, for example, water soluble polymer, water insoluble polymer, microcrystalline cellulose (for example, Avicel® PH102 or PH101), lactose in its various forms (e.g., lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.

Suitable binders include, for example, cellulose polymers (e.g., hydroxypropyl-methyl cellulose, hydroxypropylcellulose, methylcellulose and hydroxyethyl cellulose), polyvinylpyrrolidone, polyvinyl alcohol, starch or pregelatinized starch and the like or any combinations thereof.

Suitable lubricants include, for example, sodium stearyl fumarate, stearic acid, magnesium stearate, calcium stearate, zinc stearate, talc, glyceryl behenate or hydrogenated vegetable oils, and the like or any combinations thereof.

Suitable glidants can be used to improve the flowability. Suitable glidants include, for example, colloidal silica, silica gel, precipitated silica or talc, and the like or any combinations thereof.

Suitable disintegrants include, for example, sodium carboxymethyl starch, cross-linked polyvinylpyrrolidone (Crospovidone), sodium carboxymethyl glycolate (for example Explotab®), croscarmellose sodium, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.

Suitable surfactants are substances, which can lower the interfacial tension between two phases, thus enabling or supporting the formation of dispersions or working as a solubilizer. Suitable surfactants include, for example, alkyl sulfates (for example sodium lauryl sulfate), alkyltrimethylammonium salts, alcohol ethoxylates, sorbitanes (for example sorbitan laurate), polyoxyethylene sorbitanes, polyoxylglycerides, or polyoxyethylene castor oil derivatives. Sorbitan laurate and sodium lauryl sulfate are preferred surfactants.

The above disclosed unit dose formulation of the 4 APIs or the above monolithic tablet of the 2 APIs can be used as a medicament. In particular, the formulations of the present invention can also be used in the treatment of HIV-1 infection.

The present invention further provides a method for treating HIV-1 infection comprising administration of a pharmaceutically effective amount of the above unit dose formulation of the 4 APIs or of the above monolithic tablet of the 2 APIs.

The present invention is illustrated by the following examples, which are not intended to limit the scope of the invention. It will be appreciated that various modifications are within the spirit and scope of the invention.

Further aspects and embodiments of the present invention are set out in the following numbered paragraphs:
1. A pharmaceutical formulation in a unit dosage form comprising:
   (a) tenofovir or a physiologically functional derivative thereof,
   (b) emtricitabine or a physiologically functional derivative thereof,
   (c) darunavir or a physiologically functional derivative thereof and
   (d) ritonavir or a physiologically functional derivative thereof, wherein the amount of ritonavir is less than about 100 mg.
2. A pharmaceutical formulation according to Paragraph 1, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
3. A pharmaceutical formulation according to any preceding paragraph, wherein the tenofovir is in the form of its prodrug tenofovir disoproxil, or wherein the tenofovir disoproxil is in the form of its fumarate salt.
4. A pharmaceutical formulation according to any preceding paragraph, wherein the darunavir is in the form of darunavir ethanolate, hydrate, or any other crystalline form as well as amorphous darunavir, preferably, hydrate.
5. A pharmaceutical formulation according to any preceding paragraph, wherein the ritonavir is in the form of its crystalline form as well as amorphous ritonavir.
6. A pharmaceutical formulation according to any preceding paragraph, wherein the ritonavir is in the form of its crystalline form and/or amorphous ritonavir. Preferably, the ritonavir is in the form of its crystalline form or amorphous ritonavir.
7. A pharmaceutical formulation according to any preceding paragraph, wherein the ritonavir is in the form of ritonavir crystalline form II.
8. A pharmaceutical formulation according to any preceding paragraph in the form of a solid unit dosage form.
9. A pharmaceutical formulation according to any preceding paragraph for oral administration.
10. A pharmaceutical formulation according to any preceding paragraph in the form of a tablet.
11. A pharmaceutical formulation according to any preceding paragraph in the form of a film-coated tablet.
12. A pharmaceutical formulation according to any preceding paragraph wherein the pharmaceutical dosage form is a monolithic (single layer) tablet.
13. A pharmaceutical formulation according to any preceding paragraph in the form of a single unit dosage form for administration once per day.
14. A pharmaceutical formulation according to any preceding paragraph wherein the formulation is a tablet and wherein the tablet includes at least one score line.
15. A pharmaceutical formulation according to paragraph 14 wherein the tablet is provided with score line enabling the tablet to be divided into two, three or four, preferably substantially equal, parts, and preferably two substantially equal parts.
16. A pharmaceutical formulation according to any preceding paragraph wherein peak plasma concentrations of each of the active agent (a)-(c) are achieved compared with the administration of the active agents (a)-(c) as separate unit doses.
17. A pharmaceutical formulation according to any preceding paragraph wherein the peak plasma concentrations of ritonavir is less than that compared with that achieved by administration of the commercially available 100 mg ritonavir tablet composition (Norvir®).
18. A pharmaceutical formulation according to any preceding paragraph wherein the amount of ritonavir is less than or equal to about 70 mg, less than or equal to about 60 mg, less than or equal to about 50 mg.
19. A pharmaceutical formulation according to any preceding paragraph wherein the amount of ritonavir is between about 50 mg to about 60 mg, to about 70 mg, to about 80 mg, to about 100 mg, between about 60 mg to about 70 mg, to about 80 mg, to about 100 mg, between about 80 mg, to about 100 mg.
20. A pharmaceutical formulation according to any preceding paragraph wherein the amount of ritonavir is less than or equal to about 70 mg.
21. A pharmaceutical formulation according to any preceding paragraph which is chemically stable.
22. A pharmaceutical formulation according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
23. A pharmaceutical formulation according to any preceding paragraph wherein the unit dosage form has a total weight of between about 1.800 g, to about 1.900 g, to about 2.000 g.
24. A pharmaceutical formulation according to any preceding paragraph wherein the largest maximum diameter of the tablet of the present invention, comprising the 4 APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
25. A pharmaceutical formulation according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
26. A pharmaceutical formulation according to any preceding paragraph wherein the unit dosage form comprises:
   (a) about 300 mg of tenofovir disoproxil fumarate,
   (b) about 200 mg of emtricitabine,
   (c) about 800 mg of darunavir and
   (d) less than or equal to about 70 mg of ritonavir.
27. A pharmaceutical formulation according to any preceding paragraph wherein the unit dosage form comprises about 20% to about 85% by weight of active agents (a)-(d).
28. A pharmaceutical formulation according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
29. A pharmaceutical formulation according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
30. A pharmaceutical formulation according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
31. A pharmaceutical formulation according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d).
32. A pharmaceutical formulation according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
33. A pharmaceutical formulation according to any preceding paragraph wherein the unit dosage form comprises about 50%: to about 55%, to about 60%, to about 65%, to about 70%, to about 75%, to about 80%, or to about 85% by weight of active agents (a)-(d).
34. A pharmaceutical formulation according to any preceding paragraph wherein the unit dosage form comprises about 55%: to about 60%, to about 65%, to about 70%, to about 75%, to about 80%, or to about 85% by weight of active agents (a)-(d).
35. A pharmaceutical formulation according to any preceding paragraph wherein the unit dosage form comprises about 60%: to about 65%, to about 70%, to about 75%, to about 80%, or to about 85% by weight of active agents (a)-(d).
36. A pharmaceutical formulation according to any preceding paragraph wherein the unit dosage form comprises about 65%: to about 70%, to about 75%, to about 80%, or to about 85% by weight of active agents (a)-(d).
37. A pharmaceutical formulation according to any preceding paragraph wherein the unit dosage form comprises about 70%: to about 75%, to about 80%, or to about 85% by weight of active agents (a)-(d).
38. A pharmaceutical formulation according to any preceding paragraph wherein the unit dosage form comprises about 75%: to about 80%, or to about 85%, or from about 80% to about 85% by weight of active agents (a)-(d).
39. A pharmaceutical formulation according to any preceding paragraph in the form of a monolithic tablet wherein the active agents (a)-(d) are mixed and compressed to a single layer (monolithic) tablet.
40. A pharmaceutical formulation according to Paragraph 39 wherein the single layer comprises:
   (a) an admixture of: (i) a granulate of darunavir with extra-granular ritonavir or a granulate of darunavir-ritonavir, (i.e. a granulate of darunavir-ritonavir), (ii) emtricitabine granules, and (iii) tenofovir granules (preferably tenofovir disoproxil, and more preferably tenofovir disoproxil fumarate),
   (b) an admixture of (i) a granulate of darunavir with extra-granular ritonavir or a granulate of darunavir with intra-granular ritonavir, (i.e. a granulate of darunavir-ritonavir), with (ii) granules comprising emtricitabine and tenofovir; or
   (c) an admixture of: (i) a granulate of darunavir and (ii) granules comprising emtricitabine, tenofovir and ritonavir.
41. A pharmaceutical formulation according to Paragraph 40 wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
42. A pharmaceutical formulation according to Paragraph 41 wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
43. A pharmaceutical formulation according to Paragraph 40 wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
44. A pharmaceutical formulation according to Paragraph 43 wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
45. A pharmaceutical formulation according to any of Paragraphs 41 to 43 wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
46. A pharmaceutical formulation according to Paragraph 45 wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
47. A pharmaceutical formulation according to Paragraph 42 wherein the ritonavir form II is wet granulated prior to the mixing with darunavir and the other excipients, and admixing the darunavir with extra-granular ritonavir.
48. A pharmaceutical formulation according to Paragraph 47 wherein the ritonavir form II is wet granulated with at least one filler (preferably microcrystalline cellulose), followed by drying the wet granulate and milling the dry granulate.
49. A pharmaceutical formulation according to any of Paragraphs 40 to 48 wherein the granules of emtricitabine and tenofovir are prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
50. A pharmaceutical formulation according to any of Paragraphs 40-49 wherein the granules of emtricitabine, tenofovir and ritonavir are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
51. A pharmaceutical formulation according to any preceding paragraph further comprising one or more pharmaceutically acceptable excipients.
52. A pharmaceutical formulation according to Paragraph 51 wherein the pharmaceutically acceptable excipient is selected from the group consisting of fillers, binders, disintegrants, surfactants, glidants and lubricants.
53. A pharmaceutical formulation according to Paragraph 52, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
54. A pharmaceutical formulation according to Paragraph 52 or 53, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
55. A pharmaceutical formulation according to any of Paragraphs 52-54, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
56. A pharmaceutical formulation according to any of Paragraphs 52-55, wherein the glidant is selected from one or more of the group consisting of colloidal silica, silica gel, precipitated silica or talc, and the like or any combinations thereof
57. A pharmaceutical formulation according to any of Paragraphs 52-56, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
58. A pharmaceutical formulation according to any of Paragraphs 52-57, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
59. A pharmaceutical formulation according to any of Paragraphs 1-58 for use as a medicament.
60. A pharmaceutical formulation according to any of Paragraphs 1-58 for use in the treatment of HIV-1 infection.
61. A method for treating HIV-1 infection comprising administration of a pharmaceutically effective amount of the pharmaceutical formulation according to any of Paragraphs 1-58.
62. A process for preparing a pharmaceutical formulation according to any preceding paragraph, wherein the pharmaceutical formulation is a tablet, preferably a monolithic tablet, the process comprising:
   (a) blending a mixture comprising tenofovir disoproxil, emtricitabine, darunavir, ritonavir and optionally one or more pharmaceutically acceptable excipients;
   (b) compressing the blend into tablet cores; and
   (c) optionally, applying one or more coating layers over the tablet cores.
63. A process for the preparation of a pharmaceutical formulation according to any of Paragraphs 1-58 comprising combining:
   (a) darunavir with extra-granular ritonavir or darunavir with intra-granular ritonavir, (i.e. a granulate of darunavir-ritonavir),
   (b) emtricitabine granules and
   (c) tenofovir (preferably tenofovir disoproxil, and more preferably tenofovir disoproxil fumarate) granules, optionally in the presence of one or more pharmaceutically acceptable excipients to form a blend.
64. A process for the preparation of a pharmaceutical formulation according to any of Paragraphs 1-58 comprising combining:
   (a) darunavir with extra-granular ritonavir or darunavir with intra-granular ritonavir, (i.e. a granulate of darunavir-ritonavir), and
   (b) granules comprising emtricitabine and tenofovir, optionally in the presence of one or more pharmaceutically acceptable excipients to form a blend.
65. A process for the preparation of a pharmaceutical formulation according to any of Paragraphs 1-58 comprising combining:
   (a) darunavir granulate, and
   (b) granules comprising emtricitabine, tenofovir and ritonavir, optionally in the presence of one or more pharmaceutically acceptable excipients to form a blend.
66. A process according to any of Paragraph 62-65 wherein the pharmaceutically acceptable excipient comprises at least one excipient selected from a filler (preferably microcrystalline cellulose), a disintegrant (preferably crospovidone and/or croscarmellose sodium), a glidant (preferably silicon di-oxide) and a lubricant (preferably sodium stearyl fumarate).
67. A process according to Paragraph 66 wherein the pharmaceutically acceptable excipient comprises microcrystalline cellulose, crospovidone and/or croscarmellose sodium, silicon di-oxide and sodium stearyl fumarate.
68. A process according to any of Paragraphs 62-67 further comprising processing the blend to provide a solid dosage form.
69. A process according to any of Paragraphs 62-67 comprising compressing the blend to form a tablet, and optionally coating the tablet with a film coating material to form a film coated tablet.
70. A process according to Paragraph 69 wherein the blend is optionally combined with one or more pharmaceutically acceptable excipients before compressing into tablets.
71. A process according to any of Paragraphs 63-64 wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
72. A process according to Paragraph 71 wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
73. A process according to any of Paragraphs 63-64 wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
74. A process according to Paragraph 73 wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
75. A process according to either of Paragraphs 71 or 72 wherein prior to the top spray process, darunavir is mixed with at least one disintegrant such as Croscarmellose Sodium (Ac-Di-Sol®), preferably wherein the disintegrant in an amount of about 2% to about 30% by weight of total weight of the dosage form, or from about 5% to about 30%, or from about 5% to about 20%, or from about 5% to about 15%, or from about 5% to 10% by weight of total weight of the dosage form.
76. A process according to Paragraph 75 wherein the weight ratio of disintegrant (preferably croscarmellose sodium (Ac-Di-Sol®) to darunavir is from about 1:5 to about 1:25, preferably from about 1:8 to about 1:20, more preferably from about 1:10 to about 1:15.
77. A process according to Paragraph 72 wherein prior to the mixing with the granulate of darunavir and the other excipients to form darunavir with extra-granular ritonavir, the ritonavir form II is wet granulated.
78. A process according to Paragraph 77 wherein the ritonavir form II is wet granulated with at least one filler (preferably microcrystalline cellulose), followed by drying the wet granulate and milling the dry granulate.
79. A process according to any of Paragraphs 63 and 66-78, wherein the emtricitabine granules are prepared by admixing with at least one pharmaceutically acceptable excipient to form a mixture, granulating the mixture, by dry granulation, or by wet granulation followed by drying the wet granulate. 80. A process according to any of Paragraphs 63 and 66-78, wherein the emtricitabine granules are mixed with at least one filler (preferably lactose), and at least one disintegrant (preferably crospovidone) to provide a mixture, combining the mixture with a solution of a binder (preferably an aqueous solution of povidone), to form wet granules, and drying the wet granules.
81. A process according to any of Paragraphs 63 and 66-78, wherein the emtricitabine granules are prepared by combining emtricitabine with at least one filler (preferably microcrystalline cellulose and/or pregelatinized starch) and at least one disintegrant (preferably croscarmellose sodium) to obtain a mixture, combining the mixture with a solution of a binder (preferably an aqueous solution of povidone), to form wet granules, and drying the wet granules.
82. A process according to any of Paragraphs 63 and 66-81, wherein the granules of tenofovir are prepared by dry mixing, or by wet granulation followed by drying, of a mixture of tenofovir with at least one pharmaceutically acceptable excipient.
83. A process according to Paragraph 82, wherein tenofovir is dry mixed with at least one filler [preferably microcrystalline cellulose (e.g. Avicel®)], and at least one disintegrant/binder (preferably pregelatinized starch).
84. A process according to any of Paragraphs 63 and 66-81, wherein tenofovir is wet granulated with at least one filler (preferably lactose), at least one disintegrant (preferably crospovidone) and at least one binder (preferably povidone) and optionally at least one lubricant (preferably magnesium stearate), with a granulating liquid (preferably water) to provide wet granules, and drying the wet granules.
85. A process according to any of Paragraphs 63 and 66-81, wherein tenofovir is wet granulated with at least one filler (preferably microcrystalline cellulose and/or pregelatinized starch) with a granulating liquid (preferably water) to provide wet granules, and drying the wet granules.
86. A process according to any of Paragraphs 64 and 66-70, wherein the granules comprising emtricitabine and tenofovir are prepared by combining emtricitabine and tenofovir with at least one pharmaceutically acceptable excipient and wet granulating, to form wet granules, and drying the wet granules.
87. A process according to Paragraph 86, wherein emtricitabine and tenofovir are combined with at least one filler (preferably lactose), at least one disintegrant (preferably crospovidone), at least one binder (preferably pregelatinized starch or povidone) to obtain a mixture, granulating the mixture with water, and drying the granules.
88. A process according to any of Paragraphs 64 and 66-70, wherein the granules comprising emtricitabine and tenofovir are prepared by combining emtricitabine and tenofovir with at least one filler (preferably microcrystalline cellulose), at least one binder (preferably pregelatinized starch) and at least one disintegrant (preferably croscarmellose sodium) granulating the mixture with water, drying the wet granulate and milling the dry granulate.
89. A process according to any of Paragraphs 65-70, wherein the granules comprising emtricitabine, tenofovir and ritonavir form II are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
90. A process for the preparation of a pharmaceutical formulation according to any preceding paragraph further comprising one or more pharmaceutically acceptable excipients, preferably selected from one or more of the group consisting of fillers, binders, disintegrants, surfactants, glidants and lubricants.
91. A process for the preparation of a pharmaceutical formulation according to Paragraph 90, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
92. A process for the preparation of a pharmaceutical formulation according to Paragraphs 90-91, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
93. A process for the preparation of a pharmaceutical formulation according to any of Paragraphs 90-92 wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
94. A process for the preparation of a pharmaceutical formulation according to any of Paragraphs 90-93 wherein the glidant is selected from one or more of the group consisting of colloidal silica, silica gel, precipitated silica or talc, and the like or any combinations thereof.
95. A process for the preparation of a pharmaceutical formulation according to any of Paragraphs 90-94 wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof
96. A process for the preparation of a pharmaceutical formulation according to any of Paragraphs 90-95 wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
97. A pharmaceutical formulation according to Paragraph 1 wherein the ritonavir is in the form of a pre-mix, and wherein:
   at least 75% to 80% of tenofovir is released within 10 minutes
   at least 70% to 80% of emtricitabine is released within 10 minutes
   at least 25% to 30% of darunavir is released within 10 minutes, or 60% to 65% of darunavir is released within 30 minutes; and
   at least 45% to 55% of ritonavir is released within 10 minutes, or 65% to 75% of ritonavir is released within 30 minutes.
98. A pharmaceutical formulation according to any of Paragraphs 1-60 wherein at least 75% to 80% of tenofovir is released within 10 minutes.
99. A pharmaceutical formulation according to any of Paragraphs 1-60 and 98 wherein at least 70% to 80% of emtricitabine is released within 10 minutes.
100. A pharmaceutical formulation according to any of Paragraphs 1-60, and 98-99 wherein at least 25% to 30% of darunavir is released within 10 minutes, or 60% to 65% of darunavir is released within 30 minutes.
101. A pharmaceutical formulation according to any of Paragraphs 1-6, 8-41, 45-46, 49, 51-60 and 98-100 wherein the ritonavir is in the form of a premix, and wherein at least 45% to 55% of ritonavir is released within 10 minutes, or 65% to 75% of ritonavir is released within 30 minutes.
102. A pharmaceutical formulation according to any of Paragraphs 1-60 and 98-100 wherein the ritonavir is in the form of crystalline form II, and wherein at least 40% to 45% of ritonavir is released within about 10 minutes, or 70% to 75% of ritonavir is released within 30 minutes.
103. A pharmaceutical formulation according to Paragraph 1 wherein the ritonavir is in the form of crystalline form II, and wherein:
   at least 75% to 80% of tenofovir is released within 10 minutes
   at least 70% to 80% of emtricitabine is released within 10 minutes
   at least 25% to 30% of darunavir is released within 10 minutes, or 60% to 65% of darunavir is released within 30 minutes; and
   at least 40% to 45% of ritonavir is released within about 10 minutes, or 70% to 75% of ritonavir is released within 30 minutes.
104. A pharmaceutical formulation according to any of Paragraphs 1-6, 8-41, 45-46 and 51-60 wherein the ritonavir is in the form of a pre-mix, and wherein:
   at least 75% to 80% of tenofovir is released within 10 minutes
   at least 70% to 80% of emtricitabine is released within 10 minutes
   at least 25% to 30% of darunavir is released within 10 minutes, or 60% to 65% of darunavir is released within 30 minutes; and
   at least 45% to 55% of ritonavir is released within 10 minutes, or 65% to 75% of ritonavir is released within 30 minutes.
105. A pharmaceutical formulation according to any of Paragraphs 1-60 wherein the ritonavir is in the form of crystalline form II, and wherein:
   at least 75% to 80% of tenofovir is released within 10 minutes
   at least 70% to 80% of emtricitabine is released within 10 minutes
   at least 25% to 30% of darunavir is released within 10 minutes, or 60% to 65% of darunavir is released within 30 minutes; and
at least 40% to 45% of ritonavir is released within about 10 minutes, or 70% to 75% of ritonavir is released within 30 minutes.
1.A A monolithic tablet comprising:
   a) tenofovir or a physiologically functional derivative thereof,
   b) emtricitabine or a physiologically functional derivative thereof,
   c) darunavir or a physiologically functional derivative thereof and
   d) ritonavir or a physiologically functional derivative thereof, wherein the amount of ritonavir is less than or equal to about 70 mg.
2.A A monolithic tablet according to Paragraph 1A, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
3.A A monolithic tablet according to any preceding paragraph, wherein the tenofovir is in the form of its prodrug tenofovir disoproxil, or wherein the tenofovir disoproxil is in the form of its fumarate salt.
4.A A monolithic tablet according to any preceding paragraph, wherein the darunavir is in the form of darunavir ethanolate, hydrate, or any other crystalline form as well as amorphous darunavir, preferably, hydrate.
5.A A monolithic tablet according to any preceding paragraph, wherein the ritonavir is in the form of its crystalline form as well as amorphous ritonavir.
6.A A monolithic tablet according to any preceding paragraph, wherein the ritonavir is in the form of its crystalline form and/or amorphous ritonavir. Preferably, the ritonavir is in the form of its crystalline form or amorphous ritonavir.
7.A A monolithic tablet according to any preceding paragraph, wherein the ritonavir is in the form of ritonavir crystalline form II.
8.A A monolithic tablet according to any preceding paragraph in the form of a film-coated tablet.
9.A A monolithic tablet according to any preceding paragraph in the form of a single unit dosage form for administration once per day.
10.A A monolithic tablet according to any of Paragraphs 1A-9A wherein the tablet includes at least one score line.
11.A A pharmaceutical formulation according to Paragraph 10A wherein the tablet is provided with score line enabling the tablet to be divided into two, three or four, preferably substantially equal, parts, and preferably two substantially equal parts.
12.A A monolithic tablet according to any preceding paragraph wherein peak plasma concentrations of each of the active agent (a)-(c) are achieved compared with the administration of the active agents (a)-(c) as separate unit doses.
13.A A monolithic tablet according to any preceding paragraph wherein the peak plasma concentrations of ritonavir is less than that compared with that achieved by administration of the commercially available 100 mg ritonavir tablet composition (Norvir®).
14.A A monolithic tablet according to any preceding paragraph wherein the amount of ritonavir is less than or equal to about 60 mg, less than or equal to about 50 mg.
15.A A monolithic tablet according to any preceding paragraph wherein the amount of ritonavir is between about 50 mg to about 60 mg, to about 70 mg.
16.A A monolithic tablet according to any preceding paragraph which is chemically stable.
17.A A monolithic tablet according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
18.A A monolithic tablet according to any preceding paragraph wherein the unit dosage form has a total weight of between about 1.800 g, to about 1.900 g, to about 2.000 g.
19.A A monolithic tablet according to any preceding paragraph wherein the largest maximum diameter of the tablet of the present invention, comprising the four APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
20.A A monolithic tablet according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
21.A A monolithic tablet according to any preceding paragraph wherein the unit dosage form comprises:
   a) about 300 mg of tenofovir disoproxil fumarate,
   b) about 200 mg of emtricitabine,
   c) about 800 mg of darunavir and
   d) less than or equal to about 70 mg of ritonavir.
22.A A monolithic tablet according to any preceding paragraph wherein the unit dosage form comprises about 20% to about 85% by weight of active agents (a)-(d).
23.A A monolithic tablet according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
24.A A monolithic tablet according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
25.A A monolithic tablet according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
26.A A monolithic tablet according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d).
27.A A monolithic tablet according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
28.A A monolithic tablet according to any preceding paragraph wherein the unit dosage form comprises about 50%: to about 55%, to about 60%, to about 65%, to about 70%, to about 75%, to about 80%, or to about 85% by weight of active agents (a)-(d).
29.A A monolithic tablet according to any preceding paragraph wherein the unit dosage form comprises about 55%: to about 60%, to about 65%, to about 70%, to about 75%, to about 80%, or to about 85% by weight of active agents (a)-(d).
30.A A monolithic tablet according to any preceding paragraph wherein the unit dosage form comprises about 60%: to about 65%, to about 70%, to about 75%, to about 80%, or to about 85% by weight of active agents (a)-(d).
31.A A monolithic tablet according to any preceding paragraph wherein the unit dosage form comprises about 65%: to about 70%, to about 75%, to about 80%, or to about 85% by weight of active agents (a)-(d).
32.A A monolithic tablet according to any preceding paragraph wherein the unit dosage form comprises about 70%: to about 75%, to about 80%, or to about 85% by weight of active agents (a)-(d).
33.A A monolithic tablet according to any preceding paragraph wherein the unit dosage form comprises about 75%: to about 80%, or to about 85%, or from about 80% to about 85% by weight of active agents (a)-(d).
34.A A monolithic tablet according to any preceding paragraph wherein the active agents (a)-(d) are mixed and compressed to a single layer (monolithic) tablet.
35.A A monolithic tablet according to Paragraph 34A wherein the single layer comprises:
   a) an admixture of: (i) a granulate of darunavir with extra-granular ritonavir or a granulate of darunavir-ritonavir, (i.e. a granulate of darunavir-ritonavir), (ii) emtricitabine granules, and (iii) tenofovir granules (preferably tenofovir disoproxil, and more preferably tenofovir disoproxil fumarate); or
   b) an admixture of: (i) a granulate of darunavir with extra-granular ritonavir or darunavir with intra-granular ritonavir, (i.e. a granulate of darunavir-ritonavir), with (ii) granules comprising emtricitabine and tenofovir; or
   c) an admixture of: (i) a granulate of darunavir and (ii) granules comprising emtricitabine, tenofovir and ritonavir.
36.A A monolithic tablet according to Paragraph 35A wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
37.A A monolithic tablet according to Paragraph 36A wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
38.A A monolithic tablet according to Paragraph 35A wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
39.A A monolithic tablet according to Paragraph 38A wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
40.A A monolithic tablet according to any of Paragraphs 36A or 38A wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
41.A A monolithic tablet according to Paragraph 40A wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
42.A A monolithic tablet according to Paragraph 37A wherein the ritonavir form II is wet granulated prior to the mixing with darunavir and the other excipients, and admixing the darunavir with extra-granular ritonavir.
43.A A monolithic tablet according to Paragraph 42A wherein the ritonavir form II is wet granulated with at least one filler (preferably microcrystalline cellulose), followed by drying the wet granulate and milling the dry granulate.
44.A A monolithic tablet according to any of Paragraph 35A-43A wherein the granules comprising emtricitabine and tenofovir are prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
45.A A monolithic tablet according to any of Paragraphs 35A-43A wherein the granules comprising emtricitabine, tenofovir and ritonavir are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
46.A A monolithic tablet according to any preceding paragraph further comprising one or more pharmaceutically acceptable excipients.
47.A A monolithic tablet according to Paragraph 46A wherein the pharmaceutically acceptable excipient is selected from the group consisting of fillers, binders, disintegrants, surfactants, glidants and lubricants.
48.A A monolithic tablet according to Paragraph 47A, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof
49.A A monolithic tablet according to Paragraph 47A or 48A, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof
50.A A monolithic tablet according to any of Paragraphs 47A-49A, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
51.A A monolithic tablet according to any of Paragraphs 47A-50A, wherein the glidant is selected from one or more of the group consisting of colloidal silica, silica gel, precipitated silica or talc, and the like or any combinations thereof.
52.A A monolithic tablet according to any of Paragraphs 47A-51A, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
53.A A monolithic tablet according to any of Paragraphs 47A-52A, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
54.A A monolithic tablet according to any of Paragraphs 1A-53A for use as a medicament.
55.A A monolithic tablet according to any of Paragraphs 1A-53A for use in the treatment of HIV-1 infection.
56.A A method for treating HIV-1 infection comprising administration of a pharmaceutically effective amount of the monolithic tablet according to any of Paragraphs 1A-53A.
1.B A monolithic tablet comprising:
   a) tenofovir or a physiologically functional derivative thereof,
   b) emtricitabine or a physiologically functional derivative thereof,
   c) darunavir or a physiologically functional derivative thereof and
   d) ritonavir or a physiologically functional derivative thereof, wherein the ritonavir is in the form of its crystalline form II.
2.B A monolithic tablet according to Paragraph 1B, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
3.B A monolithic tablet according to any preceding paragraph, wherein the tenofovir is in the form of its prodrug tenofovir disoproxil, or wherein the tenofovir disoproxil is in the form of its fumarate salt.
4.B A monolithic tablet according to any preceding paragraph, wherein the darunavir is in the form of darunavir ethanolate, hydrate, or any other crystalline form as well as amorphous darunavir, preferably, hydrate.
5.B A monolithic tablet according to any preceding paragraph in the form of a film-coated tablet.
6.B A monolithic tablet according to any preceding paragraph in the form of a single unit dosage form for administration once per day.
7.B A monolithic tablet according to any of paragraphs 1B-6B wherein the tablet includes at least one score line.
8.B A monolithic tablet according to paragraph 7B wherein the tablet is provided with score line enabling the tablet to be divided into two, three or four, preferably substantially equal, parts, and preferably two substantially equal parts.
9.B A monolithic tablet according to any preceding paragraph wherein peak plasma concentrations of each of the active agent (a)-(c) are achieved compared with the administration of the active agents (a)-(c) as separate unit doses.
10.B A monolithic tablet according to any preceding paragraph wherein the peak plasma concentrations of ritonavir is less than that compared with that achieved by administration of the commercially available 100 mg ritonavir tablet composition (Norvir®).
11.B A monolithic tablet according to any preceding paragraph wherein the amount of ritonavir is less than or equal to about 70 mg, less than or equal to about 60 mg, less than or equal to about 50 mg.
12.B A monolithic tablet according to any preceding paragraph wherein the amount of ritonavir is between about 50 mg to about 60 mg, to about 70 mg, to about 80 mg, to about 100 mg, between about 60 mg to about 70 mg, to about 80 mg, to about 100 mg, between about 80 mg, to about 100 mg.
13.B A monolithic tablet according to any preceding paragraph wherein the amount of ritonavir is less than or equal to about 70 mg.
14.B A monolithic tablet according to any preceding paragraph which is chemically stable.
15.B A monolithic tablet according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
16.B A monolithic tablet according to any preceding paragraph wherein the unit dosage form has a total weight of between about 1.800 g, to about 1.900 g, to about 2.000 g.
17.B A monolithic tablet according to any preceding paragraph wherein the largest maximum diameter of the tablet of the present invention, comprising the 4 APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
18.B A monolithic tablet according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
19.B A monolithic tablet according to any preceding paragraph wherein the unit dosage form comprises:
   a) about 300 mg of tenofovir disoproxil fumarate,
   b) about 200 mg of emtricitabine,
   c) about 800 mg of darunavir and
   d) less than or equal to about 70 mg of ritonavir.
20.B A monolithic tablet according to any preceding paragraph wherein the unit dosage form comprises about 20% to about 85% by weight of active agents (a)-(d).
21.B A monolithic tablet according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
22.B A monolithic tablet according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
23.B A monolithic tablet according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
24.B A monolithic tablet according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d).
25.B A monolithic tablet according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
26.B A monolithic tablet according to any preceding paragraph wherein the unit dosage form comprises about 50%: to about 55%, to about 60%, to about 65%, to about 70%, to about 75%, to about 80%, or to about 85% by weight of active agents (a)-(d).
27.B A monolithic tablet according to any preceding paragraph wherein the unit dosage form comprises about 55%: to about 60%, to about 65%, to about 70%, to about 75%, to about 80%, or to about 85% by weight of active agents (a)-(d).
28.B A monolithic tablet according to any preceding paragraph wherein the unit dosage form comprises about 60%: to about 65%, to about 70%, to about 75%, to about 80%, or to about 85% by weight of active agents (a)-(d).
29.B A monolithic tablet according to any preceding paragraph wherein the unit dosage form comprises about 65%: to about 70%, to about 75%, to about 80%, or to about 85% by weight of active agents (a)-(d).
30.B A monolithic tablet according to any preceding paragraph wherein the unit dosage form comprises about 70%: to about 75%, to about 80%, or to about 85% by weight of active agents (a)-(d).
31.B A monolithic tablet according to any preceding paragraph wherein the unit dosage form comprises about 75%: to about 80%, or to about 85%, or from about 80% to about 85% by weight of active agents (a)-(d).
32.B A monolithic tablet according to any preceding paragraph in the form of a monolithic tablet wherein the active agents (a)-(d) are mixed and compressed to a single layer (monolithic) tablet.
33.B A monolithic tablet according to Paragraph 32B wherein the single layer comprises:
   a) an admixture of: (i) a granulate of darunavir with extra-granular ritonavir or a granulate of darunavir-ritonavir, (i.e. a granulate of darunavir-ritonavir), (ii) emtricitabine granules, and (iii) tenofovir granules (preferably tenofovir disoproxil, and more preferably tenofovir disoproxil fumarate); or
   b) an admixture of: (i) a granulate of darunavir with extra-granular ritonavir or a granulate of darunavir with intra-granular ritonavir, (i.e. a granulate of darunavir-ritonavir), with (ii) granules comprising emtricitabine and tenofovir; or
   c) an admixture of: (i) a granulate of darunavir and (ii) granules comprising emtricitabine, tenofovir and ritonavir.
34.B A monolithic tablet according to Paragraph 33B wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
35.B A monolithic tablet according to Paragraph 34B wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
36.B A monolithic tablet according to Paragraph 33B wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
37.B A monolithic tablet according to Paragraph 36B wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
38.B A monolithic tablet according to any of Paragraphs 34B or 36B wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
39.B A monolithic tablet according to Paragraph 38B wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
40.B A monolithic tablet according to Paragraph 35B wherein the ritonavir form II is wet granulated prior to the mixing with darunavir and the other excipients, and admixing the darunavir with extra-granular ritonavir.
41.B A monolithic tablet according to Paragraph 40B wherein the ritonavir form II is wet granulated with at least one filler (preferably microcrystalline cellulose), followed by drying the wet granulate and milling the dry granulate.
42.B A monolithic tablet according to any of Paragraphs 33B-41B wherein the granules of emtricitabine and tenofovir are prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
43.B A monolithic tablet according to any of Paragraphs 33B-41B wherein the granules comprising emtricitabine, tenofovir and ritonavir are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
44.B A monolithic tablet according to any preceding paragraph further comprising one or more pharmaceutically acceptable excipients.
45.B A monolithic tablet according to Paragraph 44B wherein the pharmaceutically acceptable excipient is selected from the group consisting of fillers, binders, disintegrants, surfactants, glidants and lubricants.
46.B A monolithic tablet according to Paragraph 45B, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
47.B A monolithic tablet according to any of Paragraphs 45B-46B, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof
48.B A monolithic tablet according to any of Paragraphs 45B-47B, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
49.B A monolithic tablet according to any of Paragraphs 45B-48B, wherein the glidant is selected from one or more of the group consisting of colloidal silica, silica gel, precipitated silica or talc, and the like or any combinations thereof.
50.B A monolithic tablet according to any of Paragraphs 45B-49B, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
51.B A monolithic tablet according to any of Paragraphs 45B-50B, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
52.B A monolithic tablet according to any of Paragraphs 1B-51B for use as a medicament.
53.B A monolithic tablet according to any of Paragraphs 1B-51B for use in the treatment of HIV-1 infection.
54.B A method for treating HIV-1 infection comprising administration of a pharmaceutically effective amount of the monolithic tablet according to any of Paragraphs 1B-51B.
1.C A monolithic tablet comprising:
   a) tenofovir or a physiologically functional derivative thereof,
   b) emtricitabine or a physiologically functional derivative thereof,
   c) darunavir or a physiologically functional derivative thereof and
2.C ritonavir or a physiologically functional derivative thereof, wherein the ritonavir is in the form of its crystalline form II and/or wherein the amount of ritonavir is less than or equal to about 70 mg.
3.C A monolithic tablet according to Paragraph 1C, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
4.C A monolithic tablet according to any preceding paragraph, wherein the tenofovir is in the form of its prodrug tenofovir disoproxil, or wherein the tenofovir disoproxil is in the form of its fumarate salt.
5.C A monolithic tablet according to any preceding paragraph, wherein the darunavir is in the form of darunavir ethanolate, hydrate, or any other crystalline form as well as amorphous darunavir, preferably, hydrate.
6.C A monolithic tablet according to any preceding paragraph in the form of a film-coated tablet.
7.C A monolithic tablet according to any preceding paragraph in the form of a single unit dosage form for administration once per day.
8.C A monolithic tablet according to any of paragraphs 1C-6C wherein the tablet includes at least one score line.
9.C A monolithic tablet according to paragraph 7C wherein the tablet is provided with score line enabling the tablet to be divided into two, three or four, preferably substantially equal, parts, and preferably two substantially equal parts.
10.C A monolithic tablet according to any preceding paragraph wherein peak plasma concentrations of each of the active agent (a)-(c) are achieved compared with the administration of the active agents (a)-(c) as separate unit doses.
11.C A monolithic tablet according to any preceding paragraph wherein the peak plasma concentrations of ritonavir is less than that compared with that achieved by administration of the commercially available 100 mg ritonavir tablet composition (Norvir®).
12.C A monolithic tablet according to any preceding paragraph wherein the amount of ritonavir is less than or equal to about 70 mg, less than or equal to about 60 mg, less than or equal to about 50 mg.
13.C A monolithic tablet according to any preceding paragraph wherein the amount of ritonavir is between about 50 mg to about 60 mg, to about 70 mg.
14.C A monolithic tablet according to any preceding paragraph which is chemically stable.
15.C A monolithic tablet according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
16.C A monolithic tablet according to any preceding paragraph wherein the unit dosage form has a total weight of between about 1.800 g, to about 1.900 g, to about 2.000 g.
17.C A monolithic tablet according to any preceding paragraph wherein the largest maximum diameter of the tablet of the present invention, comprising the 4 APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
18.C A monolithic tablet according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
19.C A monolithic tablet according to any preceding paragraph wherein the unit dosage form comprises:
   a) about 300 mg of tenofovir disoproxil fumarate,
   b) about 200 mg of emtricitabine,
   c) about 800 mg of darunavir and
   d) less than or equal to about 70 mg of ritonavir.
20.C A monolithic tablet according to any preceding paragraph wherein the unit dosage form comprises about 20% to about 85% by weight of active agents (a)-(d).
21.C A monolithic tablet according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
22.C A monolithic tablet according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
23.C A monolithic tablet according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
24.C A monolithic tablet according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d). 25.C A monolithic tablet according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
26.C A monolithic tablet according to any preceding paragraph wherein the unit dosage form comprises about 50%: to about 55%, to about 60%, to about 65%, to about 70%, to about 75%, to about 80%, or to about 85% by weight of active agents (a)-(d).
27.C A monolithic tablet according to any preceding paragraph wherein the unit dosage form comprises about 55%: to about 60%, to about 65%, to about 70%, to about 75%, to about 80%, or to about 85% by weight of active agents (a)-(d).
28.C A monolithic tablet according to any preceding paragraph wherein the unit dosage form comprises about 60%: to about 65%, to about 70%, to about 75%, to about 80%, or to about 85% by weight of active agents (a)-(d).
29.C A monolithic tablet according to any preceding paragraph wherein the unit dosage form comprises about 65%: to about 70%, to about 75%, to about 80%, or to about 85% by weight of active agents (a)-(d).
30.C A monolithic tablet according to any preceding paragraph wherein the unit dosage form comprises about 70%: to about 75%, to about 80%, or to about 85% by weight of active agents (a)-(d).
31.C A monolithic tablet according to any preceding paragraph wherein the unit dosage form comprises about 75%: to about 80%, or to about 85%, or from about 80% to about 85% by weight of active agents (a)-(d).
32.C A monolithic tablet according to any preceding paragraph wherein the active agents (a)-(d) are mixed and compressed to a single layer (monolithic) tablet.
33.C A monolithic tablet according to Paragraph 31C wherein the single layer comprises:
   a) an admixture of: (i) a granulate of darunavir with extra-granular ritonavir or a granulate of darunavir-ritonavir, (i.e. a granulate of darunavir-ritonavir), (ii) emtricitabine granules, and (iii) tenofovir granules (preferably tenofovir disoproxil, and more preferably tenofovir disoproxil fumarate); or
   b) an admixture of: (i) a granulate of darunavir with extra-granular ritonavir or a granulate of darunavir with intra-granular ritonavir, (i.e. a granulate of darunavir-ritonavir), with (ii) granules comprising emtricitabine and tenofovir; or
   c) an admixture of: (i) a granulate of darunavir and (ii) granules comprising emtricitabine, tenofovir and ritonavir.
34.C A monolithic tablet according to Paragraph 32C wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
35.C A monolithic tablet according to Paragraph 33C wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
36.C A monolithic tablet according to Paragraph 32C wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
37.C A monolithic tablet according to Paragraphs 35C wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
38.C A monolithic tablet according to any of Paragraphs 33C to 35C wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
39.C A monolithic tablet according to Paragraph 37C wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
40.C A monolithic tablet according to Paragraph 34C wherein the ritonavir form II is wet granulated prior to the mixing with darunavir and the other excipients, and admixing the darunavir with extra-granular ritonavir.
41.C A monolithic tablet according to Paragraph 39C wherein the ritonavir form II is wet granulated with at least one filler (preferably microcrystalline cellulose), followed by drying the wet granulate and milling the dry granulate.
42.C A monolithic tablet according to any of Paragraphs 32C-40C wherein the granules of emtricitabine and tenofovir are prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
43.C A monolithic tablet according to any of Paragraphs 32C-40C wherein the granules of emtricitabine, tenofovir and ritonavir are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
44.C A monolithic tablet according to any preceding paragraph further comprising one or more pharmaceutically acceptable excipients.
45.C A monolithic tablet according to Paragraph 43C wherein the pharmaceutically acceptable excipient is selected from the group consisting of fillers, binders, disintegrants, surfactants, glidants and lubricants.
46.C A monolithic tablet according to Paragraph 44C, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof
47.C A monolithic tablet according to any of Paragraphs 44C-45C, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof
48.C A monolithic tablet according to any of Paragraphs 44C-46C, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
49.C A monolithic tablet according to any of Paragraphs 44C-47C, wherein the glidant is selected from one or more of the group consisting of colloidal silica, silica gel, precipitated silica or talc, and the like or any combinations thereof
50.C A monolithic tablet according to any of Paragraphs 44C-48C, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
51.C A monolithic tablet according to any of Paragraphs 44C-49C, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
52.C A monolithic tablet according to any of Paragraphs 1C-50C for use as a medicament.
53.C A monolithic tablet according to any of Paragraphs 1C-50C for use in the treatment of HIV-1 infection.
54.C A method for treating HIV-1 infection comprising administration of a pharmaceutically effective amount of the monolithic tablet according to any of Paragraphs 1C-50C.
1.D A monolithic tablet comprising:
   a) about 300 mg of tenofovir disoproxil fumarate,
   b) about 200 mg of emtricitabine,
   c) about 800 mg of darunavir and
   d) less than or equal to about 70 mg of ritonavir.
2.D A monolithic tablet according to Paragraph 1D, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
3.D A monolithic tablet according to any preceding paragraph, wherein the darunavir is in the form of darunavir ethanolate, hydrate, or any other crystalline form as well as amorphous darunavir, preferably, hydrate.
4.D A monolithic tablet according to any preceding paragraph, wherein the ritonavir is in the form of its crystalline form as well as amorphous ritonavir.
5.D A monolithic tablet according to any preceding paragraph, wherein the ritonavir is in the form of its crystalline form and/or amorphous ritonavir. Preferably, the ritonavir is in the form of its crystalline form or amorphous ritonavir.
6.D A monolithic tablet according to any preceding paragraph, wherein the ritonavir is in the form of ritonavir crystalline form II.
7.D A monolithic tablet according to any preceding paragraph in the form of a film-coated tablet.
8.D A monolithic tablet according to any preceding paragraph in the form of a single unit dosage form for administration once per day.
9.D A monolithic tablet according to any of paragraphs 1D-8D wherein the tablet includes at least one score line.
10.D A monolithic tablet according to paragraph 9D wherein the tablet is provided with score line enabling the tablet to be divided into two, three or four, preferably substantially equal, parts, and preferably two substantially equal parts.
11.D A monolithic tablet according to any preceding paragraph wherein peak plasma concentrations of each of the active agent (a)-(c) are achieved compared with the administration of the active agents (a)-(c) as separate unit doses.
12.D A monolithic tablet according to any preceding paragraph wherein the peak plasma concentrations of ritonavir is less than that compared with that achieved by administration of the commercially available 100 mg ritonavir tablet composition (Norvir®).
13.D A monolithic tablet according to any preceding paragraph wherein the amount of ritonavir is less than or equal to about 70 mg, less than or equal to about 60 mg, less than or equal to about 50 mg.
14.D A monolithic tablet according to any preceding paragraph wherein the amount of ritonavir is between about 50 mg to about 60 mg, to about 70 mg.
15.D A monolithic tablet according to any preceding paragraph which is chemically stable.
16.D A monolithic tablet according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
17.D A monolithic tablet according to any preceding paragraph wherein the unit dosage form has a total weight of between about 1.800 g, to about 1.900 g, to about 2.000 g.
18.D A monolithic tablet according to any preceding paragraph wherein the largest maximum diameter of the tablet of the present invention, comprising the 4 APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
19.D A monolithic tablet according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
20.D A monolithic tablet according to any preceding paragraph wherein the unit dosage form comprises about 20% to about 85% by weight of active agents (a)-(d).
21.D A monolithic tablet according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
22.D A monolithic tablet according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
23.D A monolithic tablet according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
24.D A monolithic tablet according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d).
25.D A monolithic tablet according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
26.D A monolithic tablet according to any preceding paragraph wherein the unit dosage form comprises about 50%: to about 55%, to about 60%, to about 65%, to about 70%, to about 75%, to about 80%, or to about 85% by weight of active agents (a)-(d).
27.D A monolithic tablet according to any preceding paragraph wherein the unit dosage form comprises about 55%: to about 60%, to about 65%, to about 70%, to about 75%, to about 80%, or to about 85% by weight of active agents (a)-(d).
28.D A monolithic tablet according to any preceding paragraph wherein the unit dosage form comprises about 60%: to about 65%, to about 70%, to about 75%, to about 80%, or to about 85% by weight of active agents (a)-(d).
29.D A monolithic tablet according to any preceding paragraph wherein the unit dosage form comprises about 65%: to about 70%, to about 75%, to about 80%, or to about 85% by weight of active agents (a)-(d).
30.D A monolithic tablet according to any preceding paragraph wherein the unit dosage form comprises about 70%: to about 75%, to about 80%, or to about 85% by weight of active agents (a)-(d).
31.D A monolithic tablet according to any preceding paragraph wherein the unit dosage form comprises about 75%: to about 80%, or to about 85%, or from about 80% to about 85% by weight of active agents (a)-(d).
32.D A monolithic tablet according to any preceding paragraph in the form of a monolithic tablet wherein the active agents (a)-(d) are mixed and compressed to a single layer (monolithic) tablet.
33.D A monolithic tablet according to Paragraph 32D wherein the single layer comprises:
   a) an admixture of: a granulate of darunavir with extra-granular ritonavir or a granulate of darunavir-ritonavir, (i.e. a granulate of darunavir-ritonavir), emtricitabine granules, and tenofovir granules (preferably tenofovir disoproxil, and more preferably tenofovir disoproxil fumarate); or
   b) an admixture of: (i) a granulate of darunavir with extra-granular ritonavir or a granulate of darunavir with intra-granular ritonavir, (i.e. a granulate of darunavir-ritonavir), with (ii) granules comprising emtricitabine and tenofovir; or
   c) an admixture of: (i) a granulate of darunavir and (ii) granules comprising emtricitabine, tenofovir and ritonavir.
34.D A monolithic tablet according to Paragraph 33D wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
35.D A monolithic tablet according to Paragraph 34D wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
36.D A monolithic tablet according to Paragraph 33D wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
37.D A monolithic tablet according to Paragraph 36D wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
38.D A monolithic tablet according to any of Paragraphs 34D or 36D wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
39.D A monolithic tablet according to Paragraph 38D wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
40.D A monolithic tablet according to Paragraph 35D wherein the ritonavir form II is wet granulated prior to the mixing with darunavir and the other excipients, and admixing the darunavir with extra-granular ritonavir.
41.D A monolithic tablet according to Paragraph 40D wherein the ritonavir form II is wet granulated with at least one filler (preferably microcrystalline cellulose), followed by drying the wet granulate and milling the dry granulate.
42.D A monolithic tablet according to any of Paragraphs 33D-41D wherein the granules of emtricitabine and tenofovir are prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
43.D A monolithic tablet according to any of Paragraphs 33D-41B wherein the granules of emtricitabine, tenofovir and ritonavir are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
44.D A monolithic tablet according to any preceding paragraph further comprising one or more pharmaceutically acceptable excipients.
45.D A monolithic tablet according to Paragraph 44D wherein the pharmaceutically acceptable excipient is selected from the group consisting of fillers, binders, disintegrants, surfactants, glidants and lubricants.
46.D A monolithic tablet according to Paragraph 45D, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
47.D A monolithic tablet according to any of Paragraphs 45D-46B, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
48.D A monolithic tablet according to any of Paragraphs 45D-47D, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
49.D A monolithic tablet according to any of Paragraphs 45D-48D, wherein the glidant is selected from one or more of the group consisting of colloidal silica, silica gel, precipitated silica or talc, and the like or any combinations thereof.
50.D A monolithic tablet according to any of Paragraphs 45D-49D, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
51.D A monolithic tablet according to any of Paragraphs 45D-50D, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
52.D A monolithic tablet according to any of Paragraphs 1D-51D for use as a medicament.
53.D A monolithic tablet according to any of Paragraphs 1D-51D for use in the treatment of HIV-1 infection.
54.D A method for treating HIV-1 infection comprising administration of a pharmaceutically effective amount of the monolithic tablet according to any of Paragraphs 1D-51D.
1.E A monolithic tablet comprising:
   a) about 300 mg of tenofovir disoproxil fumarate,
   b) about 200 mg of emtricitabine,
   c) about 800 mg of darunavir and
   d) less than or equal to about 70 mg of ritonavir, wherein the ritonavir is in the form of its crystalline form II.
2.E A monolithic tablet according to Paragraph 1E, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
3.E A monolithic tablet according to any preceding paragraph, wherein the darunavir is in the form of darunavir ethanolate, hydrate, or any other crystalline form as well as amorphous darunavir, preferably, hydrate.
4.E A monolithic tablet according to any preceding paragraph in the form of a film-coated tablet.
5.E A monolithic tablet according to any preceding paragraph in the form of a single unit dosage form for administration once per day.
6.E A monolithic tablet according to any of paragraphs 1E-5E wherein the tablet includes at least one score line.
7.E A monolithic tablet according to paragraph 6E wherein the tablet is provided with score line enabling the tablet to be divided into two, three or four, preferably substantially equal, parts, and preferably two substantially equal parts.
8.E A monolithic tablet according to any preceding paragraph wherein peak plasma concentrations of each of the active agent (a)-(c) are achieved compared with the administration of the active agents (a)-(c) as separate unit doses.
9.E A monolithic tablet according to any preceding paragraph wherein the peak plasma concentrations of ritonavir is less than that compared with that achieved by administration of the commercially available 100 mg ritonavir tablet composition (Norvir®).
10.E A monolithic tablet according to any preceding paragraph wherein the amount of ritonavir is less than or equal to about 70 mg, less than or equal to about 60 mg, less than or equal to about 50 mg.
11.E A monolithic tablet according to any preceding paragraph wherein the amount of ritonavir is between about 50 mg to about 60 mg, to about 70 mg.
12.E A monolithic tablet according to any preceding paragraph which is chemically stable.
13.E A monolithic tablet according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
14.E A monolithic tablet according to any preceding paragraph wherein the unit dosage form has a total weight of between about 1.800 g, to about 1.900 g, to about 2.000 g.
15.E A monolithic tablet according to any preceding paragraph wherein the largest maximum diameter of the tablet of the present invention, comprising the 4 APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
16.E A monolithic tablet according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
17.E A monolithic tablet according to any preceding paragraph wherein the unit dosage form comprises about 20% to about 85% by weight of active agents (a)-(d).
18.E A monolithic tablet according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
19.E A monolithic tablet according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
20.E A monolithic tablet according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
21.E A monolithic tablet according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d).
22.E A monolithic tablet according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
23.E A monolithic tablet according to any preceding paragraph wherein the unit dosage form comprises about 50%: to about 55%, to about 60%, to about 65%, to about 70%, to about 75%, to about 80%, or to about 85% by weight of active agents (a)-(d).
24.E A monolithic tablet according to any preceding paragraph wherein the unit dosage form comprises about 55%: to about 60%, to about 65%, to about 70%, to about 75%, to about 80%, or to about 85% by weight of active agents (a)-(d).
25.E A monolithic tablet according to any preceding paragraph wherein the unit dosage form comprises about 60%: to about 65%, to about 70%, to about 75%, to about 80%, or to about 85% by weight of active agents (a)-(d).
26.E A monolithic tablet according to any preceding paragraph wherein the unit dosage form comprises about 65%: to about 70%, to about 75%, to about 80%, or to about 85% by weight of active agents (a)-(d).
27.E A monolithic tablet according to any preceding paragraph wherein the unit dosage form comprises about 70%: to about 75%, to about 80%, or to about 85% by weight of active agents (a)-(d).
28.E A monolithic tablet according to any preceding paragraph wherein the unit dosage form comprises about 75%: to about 80%, or to about 85%, or from about 80% to about 85% by weight of active agents (a)-(d).
29.E A monolithic tablet according to any preceding paragraph wherein the active agents (a)-(d) are mixed and compressed to a single layer (monolithic) tablet.
30.E A monolithic tablet according to Paragraph 29E wherein the single layer comprises:
   a) an admixture of: (i) a granulate of darunavir with extra-granular ritonavir or a granulate of darunavir-ritonavir, (i.e. a granulate of darunavir-ritonavir), (i) emtricitabine granules, and (ii) tenofovir granules (preferably tenofovir disoproxil, and more preferably tenofovir disoproxil fumarate); or
   b) an admixture of: (i) a granulate of darunavir with extra-granular ritonavir or a granulate of darunavir with intra-granular ritonavir, (i.e. a granulate of darunavir-ritonavir), with (ii) granules comprising emtricitabine and tenofovir; or
   c) an admixture of: (i) a granulate of darunavir and (ii) granules comprising emtricitabine, tenofovir and ritonavir.
31.E A monolithic tablet according to Paragraphs 30E wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
32.E A monolithic tablet according to Paragraphs 31E wherein the granulate of darunavir is further mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
33.E A monolithic tablet according to Paragraphs 30E wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir, followed by drying the wet granulate and milling the dry granulate.
34.E A monolithic tablet according to Paragraphs 33E wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
35.E A monolithic tablet according to any of Paragraphs 31E or 33E wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
36.E A monolithic tablet according to Paragraph 35E wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
37.E A monolithic tablet according to Paragraph 32E wherein the ritonavir form II is wet granulated prior to the mixing with darunavir and the other excipients, and admixing the darunavir with extra-granular ritonavir.
38.E A monolithic tablet according to Paragraph 37E wherein the ritonavir form II is wet granulated with at least one filler (preferably microcrystalline cellulose), followed by drying the wet granulate and milling the dry granulate.
39.E A monolithic tablet according to any of Paragraphs 30E-38E wherein the granules of emtricitabine and tenofovir is prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
40.E A monolithic tablet according to any of Paragraphs 30E-38E wherein the granules of emtricitabine, tenofovir and ritonavir are prepared by combining emtricitabine, tenofovir and ritonavir form II with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
41.E A monolithic tablet according to any preceding paragraph further comprising one or more pharmaceutically acceptable excipients.
42.E A monolithic tablet according to Paragraph 41E wherein the pharmaceutically acceptable excipient is selected from the group consisting of fillers, binders, disintegrants, surfactants, glidants and lubricants.
43.E A monolithic tablet according to Paragraph 42E, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
44.E A monolithic tablet according to any of Paragraphs 42E-43E, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
45.E A monolithic tablet according to any of Paragraphs 42E-44E, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
46.E A monolithic tablet according to any of Paragraphs 42E-45E, wherein the glidant is selected from one or more of the group consisting of colloidal silica, silica gel, precipitated silica or talc, and the like or any combinations thereof.
47.E A monolithic tablet according to Paragraph 42E-46E, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof
48.E A monolithic tablet according to Paragraphs 42E-47E, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
49.E A monolithic tablet according to any of Paragraphs 1E-48E for use as a medicament.
50.E A monolithic tablet according to any of Paragraphs 1E-48E for use in the treatment of HIV-1 infection.
51.E A method for treating HIV-1 infection comprising administration of a pharmaceutically effective amount of the monolithic tablet according to any of Paragraphs 1E-48E.
1.F A monolithic tablet in a unit dosage form comprising:
   a. about 300 mg of tenofovir disoproxil fumarate,
   b. about 200 mg of emtricitabine,
   c. about 800 mg of darunavir and
   d. ritonavir or a physiologically functional derivative thereof, wherein the amount of ritonavir is less than about 100 mg and wherein the ritonavir is in the form of ritonavir pre-mix.
2.F A monolithic tablet according to Paragraph 1F, wherein the physiologically functional derivative is a pharmaceutically acceptable derivative selected from the group consisting of salt, enantiomer, solid state form (crystalline, semi-crystalline or amorphous), polymorph, solvate, metabolite or prodrug in the case of (a) or (b)-(d), preferably wherein the prodrug is an ester, or a pharmaceutically acceptable salt of the enantiomer, solid state form, polymorph, solvate, metabolite or prodrug (such as an ester).
3.F A monolithic tablet according to any preceding paragraph, wherein the darunavir is in the form of darunavir ethanolate, hydrate, or any other crystalline form as well as amorphous darunavir, preferably, hydrate.
4.F A monolithic tablet according to any preceding paragraph, wherein the ritonavir is in the form of amorphous ritonavir.
5.F A monolithic tablet according to any preceding paragraph, wherein the ritonavir pre-mix comprising a co-precipitate of ritonavir with a pharmaceutically acceptable carrier, and optionally other pharmaceutically acceptable excipients.
6.F A monolithic tablet according to Paragraph 5F wherein the pharmaceutically acceptable carrier is copovidone.
7.F A monolithic tablet according to Paragraph 5F or 6F wherein the pharmaceutically acceptable excipients include a solubilizer (preferably sorbitan laurate), and/or a glidant (preferably colloidal silica).
8.F A monolithic tablet according to Paragraph 7F wherein the ritonavir is in the form of a co-precipitate comprising ritonavir, copovidone, sorbitan laurate and colloidal silica.
9.F A monolithic tablet according to any of Paragraph 5F or 6F, wherein the co-precipitate of ritonavir and a carrier (preferably copovidone) contains a weight ratio of carrier:ritonavir of: about 1:2 to about 1:10, about 1:2 to about 1:7, about 1:3 to about 1:7, about 1:3 to about 1:6, or about 1:3 to about 1:5.
10.F A monolithic tablet according to any of Paragraph 5F or 6F, wherein the co-precipitate comprises the carrier, preferably copovidone, in an amount of about 5 to about 30 wt %, about 10 to about 28 wt %, about 12 to about 25 wt %, or about 15 to about 23 wt %.
11.F A monolithic tablet according to any of Paragraph 5F or 6F, wherein the co-precipitate comprises ritonavir in an amount of about 50 to about 95 wt %, about 60 to about 85 wt %, about 70 to about 85 wt %, about 70 to about 80 wt %, about 72 to about 80 wt %, or about 75 to about 77 wt %.
12.F A monolithic tablet according to any preceding paragraph in the form of a film-coated tablet.
13.F A monolithic tablet according to any preceding paragraph in the form of a single unit dosage form for administration once per day.
14.F A monolithic tablet according to any of paragraphs 1F-13F wherein the tablet includes at least one score line.
15.F A monolithic tablet according to paragraph 14F wherein the tablet is provided with score line enabling the tablet to be divided into two, three or four, preferably substantially equal, parts, and preferably two substantially equal parts.
16.F A monolithic tablet according to any preceding paragraph wherein peak plasma concentrations of each of the active agent (a)-(c) are achieved compared with the administration of the active agents (a)-(c) as separate unit doses.
17.F A monolithic tablet according to any preceding paragraph wherein the peak plasma concentrations of ritonavir is less than that compared with that achieved by administration of the commercially available 100 mg ritonavir tablet composition (Norvir®).
18.F A monolithic tablet according to any preceding paragraph wherein the amount of ritonavir is less than or equal to about 75 mg, less than or equal to about 70 mg, less than or equal to about 60 mg, less than or equal to about 50 mg.
19.F A monolithic tablet according to any preceding paragraph wherein the amount of ritonavir is between about 50 mg to about 60 mg, to about 70 mg, to about 75 mg, to about 80 mg, to about 100 mg, between about 60 mg to about 70 mg, to about 75 mg, to about 80 mg, to about 100 mg, between about 80 mg, to about 100 mg.
20.F A monolithic tablet according to any preceding paragraph wherein the amount of ritonavir is less than or equal to about 75 mg.
21.F A monolithic tablet according to any preceding paragraph which is chemically stable.
22.F A monolithic tablet according to any preceding paragraph wherein the unit dosage form has a total weight of between about 1.800 g, to about 1.900 g, to about 2.000 g, to about 2.200 g, to about 2.300 g, to about 2.400 g, to about 2.500 g, to about 2.600 g, to about 2.800 g.
23.F A monolithic tablet according to any preceding paragraph wherein the unit dosage form has a total weight of between about 1.800 g, to about 1.900 g, to about 2.000 g.
24.F A monolithic tablet according to any preceding paragraph wherein the largest maximum diameter of the tablet of the present invention, comprising the 4 APIs, is about 27 mm or less and the depth is less than about 12.5 mm; preferably, the largest maximum diameter is between about 26 mm to about 27 mm and the depth is between about 12.5 mm to about 10 mm, more preferably, the largest maximum diameter is about 25 mm and the depth can be about 11 mm.
25.F A monolithic tablet according to any preceding paragraph wherein the hardness of the tablet of the present invention, comprising the 4 APIs, can be about 75 Strong-Cobb Units (SCU) to about 20 SCU, preferably, about 55 SCU to about 25 SCU, more preferably, about 35 SCU to about 30 SCU.
26.F A monolithic tablet according to any preceding paragraph wherein the unit dosage form comprises:

a) about 300 mg of tenofovir disoproxil fumarate,
b) about 200 mg of emtricitabine,
c) about 800 mg of darunavir and
d) less than or equal to about 75 mg of ritonavir.
27.F A monolithic tablet according to any preceding paragraph wherein the unit dosage form comprises about 20% to about 85% by weight of active agents (a)-(d).
28.F A monolithic tablet according to any preceding paragraph wherein the unit dosage form comprises about 20%: to about 25%, to about 30%, to about 35%, to about 40%, to about 45%, to about 50%, to about 55%, to about 60%, to about 65%, to about 70%, to about 75%, to about 80%, or to about 85% by weight of active agents (a)-(d).
29.F A monolithic tablet according to any preceding paragraph wherein the unit dosage form comprises about 25%: to about 30%, to about 35%, to about 40%, to about 45%, to about 50%, to about 55%, to about 60%, to about 65%, to about 70%, to about 75%, to about 80%, or to about 85% by weight of active agents (a)-(d).
30.F A monolithic tablet according to any preceding paragraph wherein the unit dosage form comprises about 30%: to about 35%, to about 40%, to about 45%, to about 50%, to about 55%, to about 60%, to about 65%, to about 70%, to about 75%, to about 80%, or to about 85% by weight of active agents (a)-(d).
31.F A monolithic tablet according to any preceding paragraph wherein the unit dosage form comprises about 40%: to about 45%, to about 50%, to about 55%, to about 60%, to about 65%, to about 70%, to about 75%, to about 80%, or to about 85% by weight of active agents (a)-(d).
32.F A monolithic tablet according to any preceding paragraph wherein the unit dosage form comprises about 45%: to about 50%, to about 55%, to about 60%, to about 65%, to about 70%, to about 75%, to about 80%, or to about 85% by weight of active agents (a)-(d).
33.F A monolithic tablet according to any preceding paragraph wherein the unit dosage form comprises about 50%: to about 55%, to about 60%, to about 65%, to about 70%, to about 75%, to about 80%, or to about 85% by weight of active agents (a)-(d).
34.F A monolithic tablet according to any preceding paragraph wherein the unit dosage form comprises about 55%: to about 60%, to about 65%, to about 70%, to about 75%, to about 80%, or to about 85% by weight of active agents (a)-(d).
35.F A monolithic tablet according to any preceding paragraph wherein the unit dosage form comprises about 60%: to about 65%, to about 70%, to about 75%, to about 80%, or to about 85% by weight of active agents (a)-(d).
36.F A monolithic tablet according to any preceding paragraph wherein the unit dosage form comprises about 65%: to about 70%, to about 75%, to about 80%, or to about 85% by weight of active agents (a)-(d).
37.F A monolithic tablet according to any preceding paragraph wherein the unit dosage form comprises about 70%: to about 75%, to about 80%, or to about 85% by weight of active agents (a)-(d).
38.F A monolithic tablet according to any preceding paragraph wherein the unit dosage form comprises about 75%: to about 80%, or to about 85%, or from about 80% to about 85% by weight of active agents (a)-(d).
39.F A monolithic tablet according to any preceding paragraph wherein the active agents (a)-(d) are mixed and compressed to a single layer (monolithic) tablet.
40.F A monolithic tablet according to Paragraph 39F wherein the single layer comprises:
   a) an admixture of: (i) a granulate of darunavir with extra-granular ritonavir or a granulate of darunavir-ritonavir, (i.e. a granulate of darunavir-ritonavir), (ii) emtricitabine granules, and (ii) tenofovir granules (preferably tenofovir disoproxil, and more preferably tenofovir disoproxil fumarate); or
   b) an admixture of: (i) a granulate of darunavir with extra-granular ritonavir or a granulate of darunavir with intra-granular ritonavir, (i.e. a granulate of darunavir-ritonavir), with (ii) granules comprising emtricitabine and tenofovir; or
   c) an admixture of: (i) a granulate of darunavir and (ii) granules comprising emtricitabine, tenofovir and ritonavir.
41.F A monolithic tablet according to Paragraph 40F wherein the granulate of darunavir is prepared by wet granulation of darunavir by top spray process of at least one binder (preferably hypromellose) dissolved in a suitable liquid (preferably water) on darunavir, followed by drying the wet granulate and milling the dry granulate.
42.F A monolithic tablet according to Paragraph 41F wherein the granulate of darunavir is further mixed with ritonavir pre-mix, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
43.F A monolithic tablet according to Paragraph 40F wherein the granulate of darunavir-ritonavir is prepared by wet granulation of darunavir with ritonavir pre-mix by top spray process of at least one binder (preferably hypromellose) dissolved in a suitable liquid (preferably water), on a mixture of darunavir and ritonavir, followed by drying the wet granulate and milling the dry granulate.
44.F A monolithic tablet according to Paragraph 43F wherein the granulate of darunavir-ritonavir is mixed with at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with intra-granular ritonavir).
45.F A monolithic tablet according to any of Paragraphs 41F to 43F wherein prior to the top spray process, Darunavir is mixed with at least one disintegrant such as Croscarmellose Sodium (Ac-Di-Sol®), preferably in an amount of about 2% to about 30% by weight of total weight of the dosage form, or from about 5% to about 30%, or from about 5% to about 20%, or from about 5% to about 15%, or from about 5% to 10% by weight of total weight of the dosage form.
46.F A monolithic tablet according to Paragraph 45F wherein the weight ratio of disintegrant (preferably croscarmellose sodium (Ac-Di-Sol®) to Darunavir is from about 1:5 to about 1:25, preferably from about 1:8 to about 1:20, more preferably from about 1:10 to about 1:15.
47.F A monolithic tablet according to any of Paragraphs 42F or 43F, wherein the ritonavir is in the form of a pre-mix, wherein ritonavir pre-mix is prepared by mixing ritonavir with at least one excipient (preferably a carrier, and more preferably copovidone) in a solvent (preferably ethanol) to form a solution, and evaporating the solvent.
48.F A monolithic tablet according to any of Paragraphs 47F, wherein the ritonavir pre-mix is prepared by mixing ritonavir with at least one excipient (preferably a carrier and more preferably copovidone), optionally with at least one solubilizer (preferably sorbitan laurate) and at least one glidant (preferably silica) in a solvent (preferably ethanol), followed by removal of the solvent by evaporation to form a solid, and optionally milling the resulting solid.
49.F A monolithic tablet according to any of Paragraphs 40E-48F wherein the granules of emtricitabine and tenofovir are prepared by a process comprising combining emtricitabine and tenofovir with at least one filler (preferably microcrystalline cellulose), at least one binder (preferably pregelatinized starch) and at least one disintegrant (preferably croscarmellose sodium) wet granulating the mixture with water, drying the wet granulate and milling the dry granulate.
50.F A monolithic tablet according to any of Paragraphs 40E-49F wherein the granules of emtricitabine, tenofovir and ritonavir are prepared by combining emtricitabine, tenofovir and ritonavir pre-mix with at least one filler (preferably microcrystalline cellulose), at least one binder (preferably pregelatinized starch) and at least one disintegrant (preferably croscarmellose sodium), granulating the mixture with water, drying the wet granulate and milling the dry granulate.
51.F A monolithic tablet according to any preceding paragraph further comprising one or more pharmaceutically acceptable excipients.
52.F A monolithic tablet according to Paragraph 51F wherein the pharmaceutically acceptable excipient is selected from the group consisting of fillers, binders, disintcgrants, surfactants, glidants and lubricants.
53.F A monolithic tablet according to Paragraph 52F, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
54.F A monolithic tablet according to any of Paragraphs 51F-53F, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
55.F A monolithic tablet according to any of Paragraphs 51F-54F, wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
56.F A monolithic tablet according to any of Paragraphs 51F-55F, wherein the glidant is selected from one or more of the group consisting of colloidal silica, silica gel, precipitated silica or talc, and the like or any combinations thereof.
57.F A monolithic tablet according to any of Paragraphs 51F-56F, wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
58.F A monolithic tablet according to any of Paragraphs 51F-57F, wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
59.F A monolithic tablet according to any of Paragraphs 1F-58F for use as a medicament.
60.F A monolithic tablet according to any of Paragraphs 1F-58F for use in the treatment of HIV-1 infection.
61.F A method for treating HIV-1 infection comprising administration of a pharmaceutically effective amount of the monolithic tablet according to any of Paragraphs 1F-58F.
1.G A monolithic tablet comprising:
   a. darunavir or a physiologically functional derivative thereof (preferably darunavir hydrate or darunavir ethanolate); wherein the amount of darunavir is 800 mg and
   b. ritonavir or a physiologically functional derivative thereof; wherein the amount of ritonavir is less than or equal to about 70 mg.
2.G A monolithic tablet according to Paragraph 1G which is chemically stable.
3.G A monolithic tablet according to any of Paragraphs 1 G-2G wherein the total tablet weight is 1.300 g or lower, about 1.200 g or lower, about 1.100 g or lower.
4.G A monolithic tablet according to any of Paragraphs 1 G-3G wherein the total tablet weight is about 1.100 g, or about 1.000 g to about 1.200 g, or about 1.000 g to about 1.300 g.
5.G A monolithic tablet according to any of Paragraphs 1G-4G wherein the total tablet weight is about 1.100 g to about 1.200 g, or about 1.100 g to about 1.300 g.
6.G A monolithic tablet according to any of Paragraphs 1G-5G wherein the total tablet weight is about 1.100 g to about 1.300 g.
7.G A monolithic tablet according to any of Paragraphs 1G-6G wherein the largest maximum diameter of the pharmaceutical formulation of the present invention, comprising darunavir and ritonavir, is about 22 mm or less and the depth is less than about 9 mm; preferably, the largest maximum diameter is between about 20 mm to about 22 mm and the depth is between about 6 mm to about 9 mm.
8.G A monolithic tablet according to any of Paragraphs 1G-8G, wherein the ritonavir is in the form of ritonavir crystalline form II.
9.G A monolithic tablet according to any of Paragraphs 1 G-8G wherein the darunavir is in the form of darunavir ethanolate, hydrate, or any other crystalline form as well as amorphous darunavir, preferably, hydrate.
10.G A monolithic tablet according to any of Paragraphs 1G-9G comprising from about 20% to about 85% by weight of darunavir and ritonavir.
11.G A monolithic tablet according to any of Paragraphs 1G-10G comprising from about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, or about 20% to about 85% by weight of darunavir and ritonavir.
12.G A monolithic tablet according to any of Paragraphs 1G-11G comprising from about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, or about 25% to about 85% by weight of darunavir and ritonavir.
13.G A monolithic tablet according to any of Paragraphs 1G-12G comprising from about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, or about 30% to about 85% by weight of darunavir and ritonavir.
14.G A monolithic tablet according to any of Paragraphs 1G-13G comprising from about 40% to about 45%, about 40% to about 50%, about 40% to about 55%, about 40% to about 60%, about 40% to about 65%, about 40% to about 70%, about 40% to about 75%, about 40% to about 80%, or about 40% to about 85% by weight of darunavir and ritonavir.
15.G A monolithic tablet according to any of Paragraphs 1G-14G comprising from about 45% to about 50%, about 45% to about 55%, about 45% to about 60%, about 45% to about 65%, about 45% to about 70%, about 45% to about 75%, about 45% to about 80%, or about 45% to about 85% by weight of darunavir and ritonavir.
16.G A monolithic tablet according to any of Paragraphs 1G-15G comprising from about 50% to about 55%, about 50% to about 60%, about 50% to about 65%, about 50% to about 70%, about 50% to about 75%, about 50% to about 80%, or about 50% to about 85% by weight of darunavir and ritonavir.
17.G A monolithic tablet according to any of Paragraphs 1G-16G comprising from about 55% to about 60%, about 55% to about 65%, about 55% to about 70%, about 55% to about 75%, about 55% to about 80% or about 55% to about 85% by weight of darunavir and ritonavir.
18.G A monolithic tablet according to any of Paragraphs 1G-17G comprising from about 60% to about 65%, about 60% to about 70%, about 60% to about 75%, about 60% to about 80%, or about 60% to about 85% by weight of darunavir and ritonavir.
19.G A monolithic tablet according to any of Paragraphs 1G-18G comprising from about 65% to about 70%, about 65% to about 75%, about 65% to about 80%, or about 65% to about 85% by weight of darunavir and ritonavir.
20.G A monolithic tablet according to any of Paragraphs 1G-19G comprising from about 70% to about 75%, about 70% to about 80%, about 70% to about 85% by weight of darunavir and ritonavir.
21.G A monolithic tablet according to any of Paragraphs 1G-20G comprising from about 75% to about 80%, about 75% to about 85% by weight of darunavir and ritonavir.
22.G A monolithic tablet according to any of Paragraphs 1G-21G comprising from about 80% to about 85% by weight of darunavir and ritonavir.
23.G A monolithic tablet according to any of Paragraphs 1G-22G further comprising at least one lubricant (preferably sodium stearyl fumarate) to form a blend that is then compressed into a tablet core.
24.G The monolithic tablet according to of Paragraph 24G wherein the tablet is coated with a film coating material to produce a film coated tablet.
25.G A monolithic tablet according to any of paragraphs 1G-25G wherein the tablet includes at least one score line.
26.G A monolithic tablet according to paragraph 26G wherein the tablet is provided with score line enabling the tablet to be divided into two, three or four, preferably substantially equal, parts, and preferably two substantially equal parts.
27.G A process for the preparation of a monolithic tablet according to any of Paragraphs 1 G-27G comprising combining darunavir with extra-granular ritonavir or darunavir with intra-granular ritonavir, (i.e. a granulate of darunavir-ritonavir), optionally in the presence of one or more pharmaceutically acceptable excipients to form a blend.
28.G A process according to Paragraph 27G wherein the pharmaceutically acceptable excipient comprises at least one excipient selected from a filler (preferably microcrystalline cellulose), a disintegrant (preferably crospovidone and/or croscarmellose sodium), a glidant (preferably silicon di-oxide) and a lubricant (preferably sodium stearyl fumarate).
29.G A process according to Paragraph 28G wherein the pharmaceutically acceptable excipient comprises microcrystalline cellulose, crospovidone and/or croscarmellose sodium, silicon di-oxide and sodium stearyl fumarate.
30.G A process according to any of Paragraphs 27G-29G further comprising processing the blend to provide a solid dosage form.
31.G A process according to any of Paragraphs 27G-30G comprising compressing the blend to form a tablet, and optionally coating the tablet with a film coating material to form a film coated tablet.
32.G A process according to 27G-31G wherein the blend is optionally combined with one or more pharmaceutically acceptable excipients before compressing into tablets.
33.G A process according to any of Paragraphs 27G-32G wherein the granulate of darunavir is mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
34.G A process according to Paragraph 33G wherein the granulate of darunavir-ritonavir is prepared by wet granulation of darunavir by top spray process of at least one binder (preferably hypromellose) dissolved in a suitable liquid (preferably water) on darunavir, drying the wet granulate and milling the dry granulate.
35.G A process according to any of Paragraphs 27G-34G wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir form II, drying the wet granulate and milling the dry granulate.
36.G A process according to any of Paragraphs 34-35G wherein the granulate of darunavir-ritonavir is mixed with at least one filler (preferably microcrystalline cellulose), at least one disintegrant (preferably crospovidone), and at least one glidant (preferably silicon di-oxide), adding at least one lubricant (preferably sodium stearyl fumarate) to the mixture to obtain the final blend of darunavir-ritonavir (preferably darunavir with intra-granular ritonavir).
37.G A monolithic tablet prepared by a process according to any of Paragraphs 27G-36G.
38.G A monolithic tablet according to any of Paragraphs 1G-27G for use as a medicament.
39.G A monolithic tablet according to any of Paragraphs 1G-27G for use in the treatment of HIV-1 infection.
40.G A method for treating HIV-1 infection comprising administration of a pharmaceutically effective amount of a monolithic tablet according to any of Paragraphs 1 G-27G.
41.G A monolithic tablet according to any preceding paragraph further comprising one or more pharmaceutically acceptable excipients, preferably selected from one or more of the group consisting of fillers, binders, disintegrants, surfactants, glidants and lubricants.
42.G A monolithic tablet according to Paragraph 41G, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
43.G A monolithic tablet according to Paragraph 41G-42G, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
44.G A monolithic tablet according to any of Paragraphs 41G-43G wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
45.G A monolithic tablet according to any of Paragraphs 41G-44G wherein the glidant is selected from one or more of the group consisting of colloidal silica, silica gel, precipitated silica or talc, and the like or any combinations thereof.
46.G A monolithic tablet according to any of Paragraphs 41G-45G wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
47.G A monolithic tablet according to any of Paragraphs 41G-46G wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
1.H A monolithic tablet comprising:
   a) darunavir or a physiologically functional derivative thereof (preferably darunavir hydrate or darunavir ethanolate); wherein the amount of 800 mg and
   b) ritonavir or a physiologically functional derivative thereof; wherein the amount of ritonavir is less than or equal to about 70 mg and wherein the ritonavir is in the form of ritonavir crystalline form II.
2.H A monolithic tablet according to Paragraph 1H which is chemically stable.
3.H A monolithic tablet according to any of Paragraphs 1H-2H wherein the total tablet weight is 1.300 g or lower, about 1.200 g or lower, about 1.100 g or lower.
4.H A monolithic tablet according to any of Paragraphs 1H-3H wherein the total tablet weight is about 1.100 g, or about 1.000 g to about 1.200 g, or about 1.000 g to about 1.300 g.
5.H A monolithic tablet according to any of Paragraphs 1H-4H wherein the total tablet weight is about 1.100 g to about 1.200 g, or about 1.100 g to about 1.300 g.
6.H A monolithic tablet according to any of Paragraphs 1H-5H wherein the total tablet weight is about 1.100 g to about 1.300 g.
7.H A monolithic tablet according to any of Paragraphs 1H-6H wherein the largest maximum diameter of the pharmaceutical formulation of the present invention, comprising darunavir and ritonavir, is about 22 mm or less and the depth is less than about 9 mm; preferably, the largest maximum diameter is between about 20 mm to about 22 mm and the depth is between about 6 mm to about 9 mm.
8.H A monolithic tablet according to any of Paragraphs 1H-7H wherein the darunavir is in the form of darunavir ethanolate, hydrate, or any other crystalline form as well as amorphous darunavir, preferably, hydrate.
9.H A monolithic tablet according to any of Paragraphs 1H-8H comprising from about 20% to about 85% by weight of darunavir and ritonavir.
10.H A monolithic tablet according to any of Paragraphs 1H-9H comprising from about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, or about 20% to about 85% by weight of darunavir and ritonavir.
11.H A monolithic tablet according to any of Paragraphs 1H-10H comprising from about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, or about 25% to about 85% by weight of darunavir and ritonavir.
12.H A monolithic tablet according to any of Paragraphs 1H-11H comprising from about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, or about 30% to about 85% by weight of darunavir and ritonavir.
13.H A monolithic tablet according to any of Paragraphs 1H-12H comprising from about 40% to about 45%, about 40% to about 50%, about 40% to about 55%, about 40% to about 60%, about 40% to about 65%, about 40% to about 70%, about 40% to about 75%, about 40% to about 80%, or about 40% to about 85% by weight of darunavir and ritonavir.
14.H A monolithic tablet according to any of Paragraphs 1H-13H comprising from about 45% to about 50%, about 45% to about 55%, about 45% to about 60%, about 45% to about 65%, about 45% to about 70%, about 45% to about 75%, about 45% to about 80%, or about 45% to about 85% by weight of darunavir and ritonavir.
15.H A monolithic tablet according to any of Paragraphs 1H-14H comprising from about 50% to about 55%, about 50% to about 60%, about 50% to about 65%, about 50% to about 70%, about 50% to about 75%, about 50% to about 80%, or about 50% to about 85% by weight of darunavir and ritonavir.
16.H A monolithic tablet according to any of Paragraphs 1H-15H comprising from about 55% to about 60%, about 55% to about 65%, about 55% to about 70%, about 55% to about 75%, about 55% to about 80% or about 55% to about 85% by weight of darunavir and ritonavir.
17.H A monolithic tablet according to any of Paragraphs 1H-16H comprising from about 60% to about 65%, about 60% to about 70%, about 60% to about 75%, about 60% to about 80%, or about 60% to about 85% by weight of darunavir and ritonavir.
18.H A monolithic tablet according to any of Paragraphs 1H-17H comprising from about 65% to about 70%, about 65% to about 75%, about 65% to about 80%, or about 65% to about 85% by weight of darunavir and ritonavir.
19.H A monolithic tablet according to any of Paragraphs 1H-18H comprising from about 70% to about 75%, about 70% to about 80%, about 70% to about 85% by weight of darunavir and ritonavir.
20.H A monolithic tablet according to any of Paragraphs 1H-19H comprising from about 75% to about 80%, about 75% to about 85% by weight of darunavir and ritonavir.
21.H A monolithic tablet according to any of Paragraphs 1H-20H comprising from about 80% to about 85% by weight of darunavir and ritonavir.
22.H A monolithic tablet according to any of Paragraphs 1H-21H further comprising at least one lubricant (preferably sodium stearyl fumarate) to form a blend that is then compressed into a tablet core.
23.H The monolithic tablet according to Paragraph 22H wherein the tablet is coated with a film coating material to produce a film coated tablet.
24.H A monolithic tablet according to any of paragraphs 1H-23H wherein the tablet includes at least one score line.
25.H A monolithic tablet according to paragraph 24H wherein the tablet is provided with score line enabling the tablet to be divided into two, three or four, preferably substantially equal, parts, and preferably two substantially equal parts.
26.H A process for the preparation of a monolithic tablet according to any of Paragraphs 1H-25H comprising combining darunavir with extra-granular ritonavir or darunavir with intra-granular ritonavir, (i.e. a granulate of darunavir-ritonavir), optionally in the presence of one or more pharmaceutically acceptable excipients to form a blend.
27.H A process according to Paragraph 26H wherein the pharmaceutically acceptable excipient comprises at least one excipient selected from a filler (preferably microcrystalline cellulose), a disintegrant (preferably crospovidone and/or croscarmellose sodium), a glidant (preferably silicon di-oxide) and a lubricant (preferably sodium stearyl fumarate).
28.H A process according to any of Paragraphs 26H-27H wherein the pharmaceutically acceptable excipient comprises microcrystalline cellulose, crospovidone and/or croscarmellose sodium, silicon di-oxide and sodium stearyl fumarate.
29.H A process according to any of Paragraphs 26H-28H further comprising processing the blend to provide a solid dosage form.
30.H A process according to any of Paragraphs 26H-29H comprising compressing the blend to form a tablet, and optionally coating the tablet with a film coating material to form a film coated tablet.
31.H A process according to 26H-30H wherein the blend is optionally combined with one or more pharmaceutically acceptable excipients before compressing into tablets.
32.H A process according to any of Paragraphs 26H-31H wherein the granulate of darunavir is mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
33.H A process according to Paragraph 32H wherein the granulate of darunavir-ritonavir is prepared by wet granulation of darunavir by top spray process of at least one binder (preferably hypromellose) dissolved in a suitable liquid (preferably water) on darunavir, drying the wet granulate and milling the dry granulate.
34.H A process according to any of Paragraphs 26H-33H wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir form II, drying the wet granulate and milling the dry granulate.
35.H A process according to Paragraph 34H wherein the granulate of darunavir-ritonavir is mixed with at least one filler (preferably microcrystalline cellulose), at least one disintegrant (preferably crospovidone), and at least one glidant (preferably silicon di-oxide), adding at least one lubricant (preferably sodium stearyl fumarate) to the mixture to obtain the final blend of darunavir-ritonavir (preferably darunavir with intra-granular ritonavir).
36.H A monolithic tablet prepared by a process according to any of Paragraphs 26H-35H.
37.H A monolithic tablet according to any of Paragraphs 1H-25H for use as a medicament.
38.H A monolithic tablet according to any of Paragraphs 1H-25H for use in the treatment of HIV-1 infection.
39.H A method for treating HIV-1 infection comprising administration of a pharmaceutically effective amount of a monolithic tablet according to any of Paragraphs 1H-25H.
40.H A monolithic tablet according to any preceding paragraph further comprising one or more pharmaceutically acceptable excipients, preferably selected from one or more of the group consisting of fillers, binders, disintegrants, surfactants, glidants and lubricants.
41.H A monolithic tablet according to Paragraph 40H, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
42.H A monolithic tablet according to Paragraph 40H-41H, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
43.H A monolithic tablet according to any of Paragraphs 40H-42H wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof.
44.H A monolithic tablet according to any of Paragraphs 40H-43H wherein the glidant is selected from one or more of the group consisting of colloidal silica, silica gel, precipitated silica or talc, and the like or any combinations thereof.
45.H A monolithic tablet according to any of Paragraphs 40H-44H wherein the disintegrant is selected from one or more of the group consisting of sodium carboxymethyl starch, cross-linked polyvinylpyrrolidone (Crospovidone), sodium carboxymethyl glycolate (for example Explotab®), croscarmellose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
46.H A monolithic tablet according to any of Paragraphs 40H-45H wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.
1.J A monolithic tablet comprising:
   a. darunavir or a physiologically functional derivative thereof (preferably darunavir hydrate or darunavir ethanolate); wherein the amount of darunavir is 800 mg and
   b. ritonavir or a physiologically functional derivative thereof; wherein the amount of ritonavir is less than or equal to about 70 mg.
2.J A monolithic tablet according to Paragraph 1J which is chemically stable.
3.J A monolithic tablet according to any of Paragraphs 1J-2J wherein the total tablet weight is 1.300 g or lower, about 1.200 g or lower, about 1.100 g or lower.
4.J A monolithic tablet according to any of Paragraphs 1J-3J wherein the total tablet weight is about 1.100 g, or about 1.000 g to about 1.200 g, or about 1.000 g to about 1.300 g.
5.J A monolithic tablet according to any of Paragraphs 1 J-4J wherein the total tablet weight is about 1.100 g to about 1.200 g, or about 1.100 g to about 1.300 g.
6.J A monolithic tablet according to any of Paragraphs 1J-5J wherein the total tablet weight is about 1.100 g to about 1.300 g.
7.J A monolithic tablet according to any of Paragraphs 1J-6J wherein the largest maximum diameter of the pharmaceutical formulation of the present invention, comprising darunavir and ritonavir, is about 22 mm or less and the depth is less than about 9 mm; preferably, the largest maximum diameter is between about 20 mm to about 22 mm and the depth is between about 6 mm to about 9 mm.
8.J A monolithic tablet according to any of Paragraphs 1J-8J, wherein the ritonavir is in the form of ritonavir crystalline form II.
9.J A monolithic tablet according to any of Paragraphs 1 J-8J wherein the darunavir is in the form of darunavir ethanolate, hydrate, or any other crystalline form as well as amorphous darunavir, preferably, hydrate.
10.J A monolithic tablet according to any of Paragraphs 1J-9J comprising from about 20% to about 85% by weight of darunavir and ritonavir.
11.J A monolithic tablet according to any of Paragraphs 1J-10J comprising from about 20% to about 25%, about 20% to about 30%, about 20% to about 35%, about 20% to about 40%, about 20% to about 45%, about 20% to about 50%, about 20% to about 55%, about 20% to about 60%, about 20% to about 65%, about 20% to about 70%, about 20% to about 75%, about 20% to about 80%, or about 20% to about 85% by weight of darunavir and ritonavir.
12.J A monolithic tablet according to any of Paragraphs 1J-11J comprising from about 25% to about 30%, about 25% to about 35%, about 25% to about 40%, about 25% to about 45%, about 25% to about 50%, about 25% to about 55%, about 25% to about 60%, about 25% to about 65%, about 25% to about 70%, about 25% to about 75%, about 25% to about 80%, or about 25% to about 85% by weight of darunavir and ritonavir.
13.J A monolithic tablet according to any of Paragraphs 1J-12J comprising from about 30% to about 35%, about 30% to about 40%, about 30% to about 45%, about 30% to about 50%, about 30% to about 55%, about 30% to about 60%, about 30% to about 65%, about 30% to about 70%, about 30% to about 75%, about 30% to about 80%, or about 30% to about 85% by weight of darunavir and ritonavir.
14.J A monolithic tablet according to any of Paragraphs 1J-13J comprising from about 40% to about 45%, about 40% to about 50%, about 40% to about 55%, about 40% to about 60%, about 40% to about 65%, about 40% to about 70%, about 40% to about 75%, about 40% to about 80%, or about 40% to about 85% by weight of darunavir and ritonavir.
15.J A monolithic tablet according to any of Paragraphs 1J-14J comprising from about 45% to about 50%, about 45% to about 55%, about 45% to about 60%, about 45% to about 65%, about 45% to about 70%, about 45% to about 75%, about 45% to about 80%, or about 45% to about 85% by weight of darunavir and ritonavir.
16.J A monolithic tablet according to any of Paragraphs 1J-15J comprising from about 50% to about 55%, about 50% to about 60%, about 50% to about 65%, about 50% to about 70%, about 50% to about 75%, about 50% to about 80%, or about 50% to about 85% by weight of darunavir and ritonavir.
17.J A monolithic tablet according to any of Paragraphs 1J-16J comprising from about 55% to about 60%, about 55% to about 65%, about 55% to about 70%, about 55% to about 75%, about 55% to about 80% or about 55% to about 85% by weight of darunavir and ritonavir.
18.J A monolithic tablet according to any of Paragraphs 1J-17J comprising from about 60% to about 65%, about 60% to about 70%, about 60% to about 75%, about 60% to about 80%, or about 60% to about 85% by weight of darunavir and ritonavir.
19.J A monolithic tablet according to any of Paragraphs 1J-18J comprising from about 65% to about 70%, about 65% to about 75%, about 65% to about 80%, or about 65% to about 85% by weight of darunavir and ritonavir.
20.J A monolithic tablet according to any of Paragraphs 1J-19J comprising from about 70% to about 75%, about 70% to about 80%, about 70% to about 85% by weight of darunavir and ritonavir.
21.J A monolithic tablet according to any of Paragraphs 1J-20J comprising from about 75% to about 80%, about 75% to about 85% by weight of darunavir and ritonavir.
22.J A monolithic tablet according to any of Paragraphs 1J-21J comprising from about 80% to about 85% by weight of darunavir and ritonavir.
23.J A monolithic tablet according to any of Paragraphs 1J-22J further comprising at least one lubricant (preferably sodium stearyl fumarate) to form a blend that is then compressed into a tablet core.
24.J The monolithic tablet according to of Paragraph 23J wherein the tablet is coated with a film coating material to produce a film coated tablet.
25.J A monolithic tablet according to any of paragraphs 1J-24J wherein the tablet includes at least one score line.
26.J A monolithic tablet according to paragraph 25J wherein the tablet is provided with score line(s) enabling the tablet to be divided into two, three or four, preferably substantially equal, parts, and preferably two substantially equal parts.
27.J A process for the preparation of a monolithic tablet according to any of Paragraphs 1 J-26J comprising combining darunavir with extra-granular ritonavir or darunavir with intra-granular ritonavir, (i.e. a granulate of darunavir-ritonavir), optionally in the presence of one or more pharmaceutically acceptable excipients to form a blend.
28.J A process according to Paragraph 27J wherein the pharmaceutically acceptable excipient comprises at least one excipient selected from a filler (preferably microcrystalline cellulose), a disintegrant (preferably crospovidone and/or croscarmellose sodium), a glidant (preferably silicon di-oxide) and a lubricant (preferably sodium stearyl fumarate).
29.J A process according to Paragraph 28J wherein the pharmaceutically acceptable excipient comprises microcrystalline cellulose, crospovidone and/or croscarmellose sodium, silicon di-oxide and sodium stearyl fumarate.
30.J A process according to any of Paragraphs 27J-29J further comprising processing the blend to provide a solid dosage form.
31.J A process according to any of Paragraphs 27J-30J comprising compressing the blend to form a tablet, and optionally coating the tablet with a film coating material to form a film coated tablet.
32.J A process according to 27J-31J wherein the blend is optionally combined with one or more pharmaceutically acceptable excipients before compressing into tablets.
33.J A process according to any of Paragraphs 27J-32J wherein the granulate of darunavir is mixed with ritonavir form II, at least one filler (preferably microcrystalline cellulose), at least one disintegrant (preferably crospovidone), and at least one glidant (preferably silicon di-oxide), followed by addition of at least one lubricant (preferably sodium stearyl fumarate) to the obtained mixture to obtain the final blend of darunavir-ritonavir (preferably darunavir with extra-granular ritonavir).
34.J A process according to Paragraph 33J wherein the granulate of darunavir-ritonavir is prepared by wet granulation of darunavir by top spray process of at least one binder (preferably hypromellose) dissolved in a suitable liquid (preferably water) on darunavir, drying the wet granulate and milling the dry granulate.
35.J A process according to any of Paragraphs 27J-34J wherein the granulate of darunavir-ritonavir is prepared by wet granulation of darunavir with ritonavir form II by top spray process of at least one binder (preferably hypromellose) dissolved in a suitable liquid (preferably water), on a mixture of darunavir and ritonavir form II, drying the wet granulate and milling the dry granulate.
36.J A process according to any of Paragraphs 34J-35J wherein the granulate of darunavir-ritonavir is mixed with at least one filler (preferably microcrystalline cellulose), at least one disintegrant (preferably crospovidone), and at least one glidant (preferably silicon di-oxide), adding at least one lubricant (preferably sodium stearyl fumarate) to the mixture to obtain the final blend of darunavir-ritonavir (preferably darunavir with intra-granular ritonavir).
37.J A monolithic tablet prepared by a process according to any of Paragraphs 27J-36J.
38.J A monolithic tablet according to any of Paragraphs 1J-26J for use as a medicament.
39.J A monolithic tablet according to any of Paragraphs 1J-26J for use in the treatment of HIV-1 infection.
40.J A method for treating HIV-1 infection comprising administration of a pharmaceutically effective amount of a monolithic tablet according to any of Paragraphs 1 J-26J.
41.J A monolithic tablet according to any preceding paragraph further comprising one or more pharmaceutically acceptable excipients, preferably selected from one or more of the group consisting of fillers, binders, disintegrants, surfactants, glidants and lubricants.
42.J A monolithic tablet according to Paragraph 41J, wherein the filler (diluent) is selected from one or more of the group consisting of water soluble polymer, water insoluble polymer, microcrystalline cellulose (for example, Avicel® PH102 or PH101), lactose in its various forms (e.g. lactose USP, anhydrous or spray dried), sorbitol, dextrose, sucrose, mannitol, xylitol, maltose, polyols, fructose, guar gum, magnesium hydroxide, dicalcium phosphate, anhydrous calcium hydrogen phosphate, starch, and the like or any combinations thereof.
43.J A monolithic tablet according to Paragraph 41 J-42J, wherein the binder is selected from one or more of the group consisting of cellulose polymers (e.g. hydroxypropylmethyl cellulose, hydroxypropylcellulose, methylcellulose and hydroxyethyl cellulose), polyvinylpyrrolidone, polyvinyl alcohol, starch or pregelatinized starch and the like or any combinations thereof.
44.J A monolithic tablet according to any of Paragraphs 41J-43J wherein the lubricant is selected from one or more of the group consisting of sodium stearyl fumarate, stearic acid, magnesium stearate, calcium stearate, zinc stearate, talc, glyceryl behenate or hydrogenated vegetable oils, and the like or any combinations thereof
45.J A monolithic tablet according to any of Paragraphs 41 J-44J wherein the glidant is selected from one or more of the group consisting of colloidal silica, silica gel, precipitated silica or talc, and the like or any combinations thereof.
46.J A monolithic tablet according to any of Paragraphs 41J-45J wherein the disintegrant is selected from one or more of the group consisting of sodium carboxymethyl starch, cross-linked polyvinylpyrrolidone (Crospovidone), sodium carboxymethyl glycolate (for example Explotab®), croscarmcllose, swelling polysaccharide, for example soy polysaccharide, carrageenan, agar, pectin, starch and derivatives thereof, protein, for example formaldehyde-casein, sodium bicarbonate, ion exchange resin and the like or any combinations thereof.
47.J A monolithic tablet according to any of Paragraphs 41J-46J wherein the surfactant is selected from one or more of the group consisting of alkyl sulfates (particularly sodium lauryl sulfate), alkyltrimethylammonium salts, alcohol ethoxylates, sorbitans (particularly sorbitan laurate), polyoxyethylene sorbitans, polyoxylglycerides, or polyoxyethylene castor oil derivatives, and preferably wherein the surfactant is selected from one or more of sorbitan laurate and sodium lauryl sulfate.

### ANALYTICAL METHOD

### Assay Analytical Method for Tablet Comprising Darunavir and Ritonavir

### Chromatographic System

Column & Packing: Phenomenex Luna C18(2) 5 μm,100A
   250×4.6 mm
Column Temperature: 30
Mobile Phase: Buffer sol. (pH 3.2):Acetonitrile (40:60)
Flow Rate: 1.0 mL/min.
Detector: UV at 240 nm, 10 mm flow cell path length

### Analytical Method for Tablet Comprising Emtricitabine, Tenofovir, Darunavir, Ritonavir:

### Impurities and Degradants Determination Chromatographic Conditions for Emtricitabine/Tenofovir/Darunavir:

### Column: Inertsil ODS-3, 150×4.6 mm, 3 um

Column temperature: 40° C.
Mobile phase:
Sol. A: Acetate buff. pH 4.6:MeOH (95:5 v/v)
Sol. B: acetonitrile (ACN)
By gradient:
Flow: 0.8 mL/min

### Detector: UV at 262 nm (by PDA),

10 mm flow cell path length

### Assay Chromatographic Conditions:

### Column: Phenomenex Luna C18(2),

250×4.6 mm, 5 um
Column temperature: 30° C.
Mobile phase:
Sol. A: H₂O (pH 3.2):ACN (90:10 v/v)
Sol. B: ACN
By gradient:
Detector: UV at 240 nm, 10 mm flow cell
path length

Gradient

| Time | Sol. A | Sol. B |
|---|---|---|
| 0 | 99.0 | 1.0 |
| 10 | 95.0 | 5.0 |
| 15 | 90.0 | 10.0 |
| 59 | 29.0 | 71.0 |
| 60 | 1.0 | 99.0 |
| 62 | 1.0 | 99.0 |
| 62.1 | 99.0 | 1.0 |
| 68 | 99.0 | 1.0 |

### Impurities and Degradants Determination Chromatographic Conditions for Ritonavir.

### Column: ACQUITY UPLC BEH C8 1.7 um, 2.1×100 mm

Column temperature: 50° C.
Mobile phase:
Sol. A=Buffer: tetrahydrofuran (THF):n-Butanol (87:8:5 v/v)
Sol. B=ACN:THF:n-Butanol (87:8:5 v/v)
By gradient:
Flow: 0.6 mL/min

### Detector: UV at 240 nm (by PDA),

10 mm flow cell path length

Gradient

| Time | Sol. A | Sol. B |
|---|---|---|
| 0 | 85 | 15 |
| 10 | 75 | 25 |
| 12 | 75 | 25 |
| 15 | 30 | 70 |
| 16 | 30 | 70 |
| 16.1 | 85 | 15 |
| 20 | 85 | 15 |

### Release Rate Determination for Darunavir/Ritonavir/Tenofovir/Emtricitabine

As used herein, the dissolution profile (drug release rate) was determined using 1800 ml of 2% Brij-35 (30% solution of nonionic polyoxyethylene surfactant in water) in 0.05M phosphate buffer, having a pH of 3.0, using paddles apparatus (USP apparatus II) at 75 rpm, at a temperature of 37° C.±0.5° C. In particular the following procedure and conditions were employed.
Equipment: 6-vessel assembly, Apparatus 2 (Paddles)
Medium*: 1.5% Brij-35 (nonionic polyoxyethylene surfactant) in water (30% solution)

### Volume: 1800 mL

### Stirring Rate: 75 rpm

### Temperature: 37° C.±0.5° C.

Sampling time:

### For Darunavir/Tenofovir/Emtricitabine: 10, 20, 30, 45 min.

### For Ritonavir: 10, 20, 30, 45, 90, 120 min.

Sampling volume: 4 mL
Chromatographic conditions:

### Column: ZORBAX SB C8, 75×4.6 mm, 3.5 μm

Column temperature: 30° C.
Mobile phase:

Sol. A=0.01% Formic Acid: Acetonitrile (95:5v/v)

Sol. B=Acetonitrile

By gradient:

Dissolution Gradient

| Time | Sol. A | Sol. B |
|---|---|---|
| 0 | 100 | 0 |
| 8 | 35 | 65 |
| 9 | 35 | 65 |
| 9.5 | 100 | 0 |
| 11 | 100 | 0 |

Flow: 2.0 mL/min
Detector: UV at 230 nm, 10 mm flow cell path length
Injection volume: 10 uL
Injector wash: Acetonitrile 100%
Autosampler temperature: 8° C.
Dissolution Working standard solutions:
Emtricitabine concentration: 0.11 mg/mL in dissolution medium.
Tenofovir concentration: 0.17 mg/mL in dissolution medium.
Darunavir concentration: 0.45 mg/mL in dissolution medium.
Ritonavir concentration: 0.038 mg/mL in dissolution medium.

### EXAMPLES

### Example 1

### Preparation of Ritonavir Pre-Mix

| | A | B | C |
|---|---|---|---|
| Ingredient | mg/tablet | mg/tablet | mg/tablet |
| Ritonavir | 100.00 | 102.41 | 71.7 |
| Copovidone | 20.00 | 30.7 | 21.5 |
| Sorbitan laurate | 7.00 | 3.06 | 2.14 |
| Colloidal silica, anhydrous | 3.00 | 0.041 | 0.028 |
| Pre-mix total: | 130.00 | 136.24 | 95.37 |

Ritonavir and the excipients were dissolved in ethanol. Ethanol was evaporated to obtain dry powder. The powder was then milled to lower particle size

### Example 2

### Preparation of a Monolithic Tablet Containing Darunavir-Ritonavir Extra-Granular

| Raw Material | Mg/tablet |
|---|---|
| Darunavir Hydrate | 865.75 |
| Hypromellose (Methocel ® E-15) | 12.20 |
| microcrystalline cellulose (Avicel 101) | 14.00 |
| Ex-Granular (Part I) | |
| microcrystalline cellulose (Avicel 102) | 150.26 |
| Crospovidone LX | 30.50 |
| Silicon di-oxide (Aerosil) | 3.04 |
| Ritonavir Pre-mix (example 1) | 130.00 |
| Ex-Granular (Part II) | |
| Sodium Stearyl Fumarate | 12.00 |
| Total Tablet weight | 1217.75 |

Darunavir granulation: darunavir was wet granulated by a top spray process using hypromellose (Methocel® E-15) as granulation solution and purified water as granulation liquid.

The wet granulated material was then dried in Fluid bed drier and milled to lower particle size.

Darunavir granulate, ritonavir pre-mix (prepared according to example 1), crospovidone, microcrystalline cellulose (Avicel® 102) and Silicon di-oxide (Aerosil) were mixed and then sodium stearyl fumarate was added for final mixing, and the mixture was further compressed into tablet cores.

The tablet cores were then coated with a film coating material to produce film coated tablets. (Approximately 3% weight gain).

### Example 3

### Preparation of a Monolithic Tablet Containing Darunavir-Ritonavir (Intra-Granular)

| Raw Material | mg/tablet |
|---|---|
| Darunavir Hydrate | 865.75 |
| Hypromellose (Methocel ® E-15) | 12.20 |
| Ritonavir Pre-mix (Example 1) | 130.00 |
| Ex-Granular (Part I) | |
| microcrystalline cellulose (Avicel ® 102) | 164.26 |
| Crospovidone LX | 30.50 |
| Silicon di-oxide (Aerosil ®) | 3.05 |
| Ex-Granular (Part II) | |
| Sodium stearyl fumarate | 12.00 |
| Total Tablet weight | 1217.76 |

Darunavir-Ritonavir granulation: darunavir was wet granulated with ritonavir pre-mix (prepared according to example 1) by a top spray process using hypromellose (Methocel E-15) as granulation solution and purified water as granulation liquid.

The wet granulated material was then dried in Fluid bed drier and milled to lower particle size.

Darunavir-ritonavir granulate, crospovidone, microcrystalline cellulose (Avicel 102) and Silicon di-oxide (Aerosil) were mixed and then sodium stearyl fumarate was added for final mixing, and the mixture was further compressed into tablet cores.

The tablet cores were then coated with a film coating material to produce film coated tablets. (Approximately 3% weight gain).

### Example 4

### Preparation of Emtricitabine/Tenofovir Granulate

| | A | B |
|---|---|---|
| Raw Material | mg/tablet | mg/tablet |
| Emtricitabine | 200 | 200 |
| Tenofovir disoproxil fumarate | 300 | 300 |
| microcrystalline cellulose (Avicel ® 102) | 90.30 | 90.30 |
| Lactose monohydrate | 48.10 | 48.10 |
| Pregelatinized starch 1500 | 30.09 | 30.09 |
| Croscarmellose Sodium (Ac-Di-Sol ®) | 3.51 | 3.51 |
| Ex-Granular (Part I) | | |
| Croscarmellose Sodium (Ac-Di-Sol ®) | 28.0 | |
| Total weight | 700.00 | 672.00 |

Emtricitabine/tenofovir disoproxil fumarate were wet granulated by High Shear Mixer with microcrystalline cellulose (Avicel® 102), Lactose monohydrate, Pregelatinized Starch and Croscarmellose Sodium (Ac-Di-Sol®). The granules were then dried by Fluid Bed Drier and milled to lower particle size. Optionally, croscarmellose sodium was then added as an extra-granular disintegrant.

### Example 5

### Preparation of Emtricitabine Granulate

| Ingredient | mg/tablet |
|---|---|
| Emtricitabine | 200.00 |
| Mannitol | 50.00 |
| Lactose monohydrate | 90.00 |
| Crospovidone | 30.00 |
| Povidone | 10.00 |
| Emtricitabine granulate total: | 380.00 |

Emtricitabine is mixed with excipients and wet granulated using povidone solution in purified water. The wet granulate is then dried in a fluid bed dryer.

### Example 5A

### Preparation of Emtricitabine Granulate

| Ingredient | mg/tablet |
|---|---|
| Emtricitabine | 200.00 |
| Microcrystalline cellulose (Avicel pH 101) | 25.00 |
| Croscarmellose Sodium (Ac-Di-Sol ®) | 25.00 |
| Pregelatinized starch 1500 | 50.00 |
| Emtricitabine granulate total: | 350.00 |

Emtricitabine was mixed with excipients and wet granulated using povidone solution in purified water. The wet granulate was then dried in a fluid bed dryer.

### Example 6

### Preparation of Tenofovir Disoproxil Fumarate Granulate

| Ingredient | mg/tablet |
|---|---|
| Tenofovir Disoproxil Fumarate | 300.00 |
| Microcrystalline cellulose (Avicel PH 101) | 130.00 |
| Lactose monohydrate | 100.00 |
| Pregelatinized starch | 50.00 |
| Tenofovir granulate total: | 580.00 |

Tenofovir Disoproxil Fumarate was mixed with excipients and wet granulated using purified water. The wet granulate was then dried in a fluid bed dryer.

### Example 7

### Preparation of Tenofovir Disoproxil Fumarate Granulate by Dry Mixing

| Ingredient | mg/tablet |
|---|---|
| Tenofovir disoproxil fumarate | 300.00 |
| Mannitol | 45.00 |
| Lactose monohydrate | 144.00 |
| Crospovidone | 36.00 |
| Total weight: | 525.00 |

Tenofovir granulation is produced by dry mixing of tenofovir Disoproxil together with the excipients.

### Example 7A

### Preparation of Tenofovir Disoproxil Fumarate Granulate

| Ingredient | mg/tablet |
|---|---|
| Tenofovir Disoproxil Fumarate | 300.00 |
| Microcrystalline cellulose (Avicel PH 101) | 230.00 |
| Pregelatinized starch | 50.00 |
| Tenofovir granulate total: | 580.00 |

Tenofovir Disoproxil Fumarate was mixed with excipients and wet granulated using purified water. The wet granulate was then dried in a fluid bed dryer.

### Example 8

### Preparation of a Monolithic Tablet Containing Emtricitabine, Tenofovir, Darunavir and Ritonavir (Extra-Granular)

| Raw Material | mg/tablet A | mg/tablet B |
|---|---|---|
| Emtricitabine/Tenofovir disoproxil | 700.00 | 672.00 |
| fumarate Granulate including the extra- | | |
| granular disintegrant, prepared according | | |
| to example 4A or 4B | | |
| Darunavir - Ritonavir extra-granular, | 1217.75 | 1217.75 |
| prepared according to example 2, | | |
| excluding the compression step. | | |
| Total weight | 1917.75 | 1889.75 |

Emtricitabine/tenofovir granulate including the extra-granular disintegrant, was prepared according to Example 4A or 4B. Then darunavir-ritonavir formulations, prepared according to Example 2, were added. Then, the final mixture was further compressed into tablet cores.

The tablet cores were then coated with a film coating material to produce film coated tablets. (Approximately 3% weight gain).

### Example 9

### Preparation of a Bi-Layer Tablet Containing Emtricitabine and Tenofovir in the First Layer, Darunavir and Ritonavir (Extra-Granular) in the Second Layer

| Raw Material | mg/tablet |
|---|---|
| Emtricitabine/Tenofovir Disoproxil fumarate | 700.00 |
| Granulate including the extra-granular disintegrant, | |
| prepared according to example 4A | |
| Magnesium stearate | 7.00 |
| Darunavir - Ritonavir extra-granular, prepared | 1217.75 |
| according to example 2, excluding the compression | |
| step. | |
| Total weight | 1924.75 |

### Final Blend 1^{st} Layer:

Emtricitabine/tenofovir granulate including the extra-granular disintegrant is prepared according to Example 4A, then it is mixed with Magnesium stearate.

### Final Blend 2^{nd} Layer:

Darunavir-ritonavir formulation, prepared according to Example 2, excluding the compression step.

The two blends are then compressed into tablet cores.

The tablet cores are then coated with a film coating material to produce film coated tablets. (Approximately 3% weight gain).

### Example 10

### Preparation of a Monolithic Tablet Containing Emtricitabine, Tenofovir, Darunavir and Ritonavir (Intra-Granular)

| Raw Material | mg/tablet | mg/tablet B |
|---|---|---|
| Emtricitabine/Tenofovir Disoproxil | 700.00 | 672.00 |
| fumarate Granulate including the extra- | | |
| granular disintegrant addition, prepared | | |
| according to example 4A or 4B. | | |
| Darunavir - Ritonavir intra-granular, | 1217.76 | 1217.76 |
| prepared according to example 3, excluding | | |
| the compression step | | |
| Total weight | 1917.76 | 1889.76 |

Emtricitabine/tenofovir granulate including the extra-granular disintegrant, was prepared according to Example 4A or 4B. Then darunavir-ritonavir formulation, prepared according to Example 3 was added, excluding the compression step. Then, the final mixture was further compressed into tablet cores.

The tablet cores were then coated with a film coating material to produce film coated tablets. (Approximately 3% weight gain).

### Example 11

### Preparation of a Bi-Layer Tablet Containing Emtricitabine and Tenofovir in the First Layer, Darunavir and Ritonavir (Intra-Granular) in the Second Layer

| Raw Material | mg/tablet |
|---|---|
| Emtricitabine/Tenofovir Disoproxil | 700.00 |
| fumarate Granulate, prepared according to | |
| Example 4A | |
| Magnesium stearate | 7.00 |
| Darunavir - Ritonavir intra-granular, | 1217.76 |
| prepared according to Example 3, excluding | |
| the compression step | |
| Total weight | 1924.76 |

Final Blend 1^{st} Layer:

Emtricitabine/tenofovir granulate including the extra-granular disintegrant, is prepared according to Example 4A, then it is mixed with magnesium stearate.

### Final Blend 2^{nd} Layer:

Darunavir-ritonavir formulation, prepared according to Example 3, excluding the compression step.

The two blends are then compressed into tablet cores.

The tablet cores are then coated with a film coating material to produce film coated tablets. (Approximately 3% weight gain).

### Example 12

### Preparation of a Monolithic Tablet Containing Darunavir-Ritonavir Extra-Granular

| | Exp 12A | Exp 12B |
|---|---|---|
| Raw Material | mg/tablet | mg/tablet |
| Darunavir Hydrate | 865.75 | 865.75 |
| Hypromellose (Methocel ® E-15) | 12.20 | 12.20 |
| Croscarmellose Sodium (Ac-Di-Sol ®) | 60.99 | 60.99 |
| Purified Water | | |
| Ex-Granular (Part I) | | |
| Microcrystalline cellulose (Avicel ® 102) | 99.71 | 99.71 |
| Crospovidone LX | 35.00 | 35.00 |
| Silicon di-oxide (Aerosil) | 3.04 | 3.04 |
| Ritonavir Pre-mix (Example 1B; Xxample 1C) | 136.24 | 95.37 |
| Ex-Granular (Part II) | | |
| Sodium stearyl fumarate | 13.00 | 13.00 |
| Total Tablet weight | 1225.93 | 1185.06 |

Darunavir granulation: Darunavir and Croscarmellose Sodium (Ac-Di-Sol®) were wet granulated by top spray process using Hypromellose (Methocel® E-15) as granulation solution and purified water as granulation liquid.

The wet granulated material was then dried in Fluid bed drier and milled by Quadro to desired particle size.

Darunavir granulate, ritonavir pre-mix (prepared according to example 1B and 1C), crospovidone, microcrystalline cellulose (Avicel 102) and Silicon di-oxide (Aerosil)-were mixed and then sodium stearyl fumarate was added for final mixing, and the mixture was further compressed into tablet cores.

The tablet cores were then coated with a film coating material to produce film coated tablets. (Approximately 3% weight gain).

### Example 13

### Preparation of a Monolithic Tablet Containing Darunavir-Ritonavir Intra-Granular

| | A | B |
|---|---|---|
| Raw Material | mg/tablet | mg/tablet |
| Darunavir Hydrate | 865.75 | 865.75 |
| Hypromellose (Methocel ® E-15) | 12.20 | 12.20 |
| Croscarmellose Sodium (Ac-Di-Sol ®) | 60.99 | 60.99 |
| Ritonavir Form II | 70.0 | 70.00 |
| Purified Water | | |
| Ex-Granular (Part I) | | |
| Microcrystalline cellulose (Avicel 102) | 98.08 | 98.08 |
| Crospovidone LX | 35.00 | 35.00 |
| Silicon di-oxide (Aerosil) | 3.04 | 3.04 |
| hydrogenated castor oil of (powder) | 34.95 | |
| Ex-Granular (Part II) | | |
| sodium stearyl fumarate | 13.00 | 13.00 |
| Total Tablet weight | 1193.01 | 1158.06 |

Darunavir-Ritonavir granulation: Darunavir, Ritonavir form II and Croscarmellose Sodium (Ac-Di-Sol®) were wet granulated by top spray process using Hypromellose (Methocel® E-15) as granulation solution and purified water as granulation liquid.

The wet granulated material is then dried in Fluid bed drier and milled by Quadro to desired particle size.

Darunavir granulate, crospovidone, microcrystalline cellulose (Avicel® 102), hydrogenated castor oil (optionally) and Silicon di-oxide (Aerosil®) were mixed and then sodium stearyl fumarate was added for final mixing, and the mixture was further compressed into tablet cores.

The tablet cores are then coated with a film coating material to produce film coated tablets. (Approximately 3% weight gain).

### Example 14

### Preparation of a Monolithic Tablet Containing Darunavir-Ritonavir Extra-Granular

| | A | B |
|---|---|---|
| Raw Material | mg/tablet | mg/tablet |
| Darunavir Hydrate | 865.75 | 865.75 |
| Hypromellose (Methocel ® E-15) | 12.20 | 12.20 |
| Croscarmellose Sodium (Ac-Di-Sol ®) | 60.99 | 60.99 |
| Purified Water | | |
| Ex-Granular (Part I) | | |
| Microcrystalline cellulose (Avicel ® 102) | 98.08 | 98.08 |
| Crospovidone LX | 35.00 | 35.00 |
| Silicon di-oxide (Aerosil) | 3.04 | 3.04 |
| hydrogenated castor oil of (powder) | 34.95 | |
| Ritonavir Form II | 70.0 | 70.00 |
| Ex-Granular (Part II) | | |
| Sodium stearyl fumarate | 13.00 | 13.00 |
| Total Tablet weight | 1193.01 | 1158.06 |

Darunavir-Ritonavir granulation: Darunavir, Ritonavir form II and Croscarmellose Sodium (Ac-Di-Sol®) were wet granulated by top spray process using Hypromellose (Methocel® E-15) as granulation solution and purified water as granulation liquid.

The wet granulated material is then dried in Fluid bed drier and milled by Quadro to desired particle size.

Darunavir granulate, crospovidone, microcrystalline cellulose (Avicel® 102), optionally, hydrogenated castor oil and Silicon di-oxide (Aerosil®) were mixed and then sodium stearyl fumarate was added for final mixing, and the mixture was further compressed into tablet cores.

The tablet cores are then coated with a film coating material to produce film coated tablets. (Approximately 3% weight gain).

### Example 15

### Preparation of a Monolithic Tablet Containing Emtricitabine, Tenofovir, Darunavir and Ritonavir (Intra/Extra-Granular)

| | Exp 15A | Exp 15B |
|---|---|---|
| Raw Material | mg/tablet | mg/tablet |
| Emtricitabine/Tenofovir disoproxil | 700.00 | 700.00 |
| fumarate Granulate including the extra- | | |
| granular disintegrant, prepared according | | |
| to example 4 | | |
| Darunavir - Ritonavir intra/extra-granular, | 1193.01 | 1158.06 |
| prepared according to example 13 or 14, | | |
| excluding the compression step. | | |
| Total weight | 1893.01 | 1858.06 |

Emtricitabine/Tenofovir granulate including the extra-granular disintegrant, was prepared according to Example 4. Then Darunavir-Ritonavir formulations, prepared according to Example 13 or 14, were added. Then, the final mixture was further compressed into tablet cores.

The tablet cores were then coated with a film coating material to produce film coated tablets. (Approximately 3% weight gain).

### Example 16

### Preparation of a Monolithic Tablet Containing Darunavir-Ritonavir Extra-Granular

| | |
|---|---|
| Raw Material | mg/tablet |

Darunavir Granulate

| | |
|---|---|
| Darunavir Hydrate | 865.75 |
| Hypromellose (Methocel E-15) | 12.20 |
| Croscarmellose Sodium (Ac-Di-Sol ®) | 60.99 |
| Purified Water | |

Ritonavir Granulate

| | |
|---|---|
| Ritonavir Form II | 70.0 |
| Microcrystalline cellulose (Avicel ® 101) | 30.0 |
| Purified Water | |
| Ex-Granular (Part I) | |
| Microcrystalline cellulose (Avicel ® 102) | 68.08 |
| Crospovidone LX | 35.00 |
| Silicon di-oxide (Aerosil) | 3.04 |
| Ex-Granular (Part II) | |
| Sodium stearyl fumarate | 13.00 |
| Total Tablet weight | 1158.06 |

Darunavir granulation: Darunavir, and Croscarmellose Sodium (Ac-Di-Sol®) were wet granulated by top spray process using Hypromellose (Methocel^{e} E-15) as granulation solution and purified water as granulation liquid.

The wet granulated material was then dried in Fluid bed drier and milled by Quadro to desired particle size.

Ritonavir granulation-Ritonavir and microcrystalline cellulose (Avicel® 101) were wet granulated by high shear mixer using purified water as granulation liquid.

The wet granulated material was then dried in Fluid bed drier and milled by Quadro to desired particle size.

Darunavir granulate and Ritonavir granulate, crospovidone, microcrystalline cellulose (Avicel® 102), and Silicon di-oxide (Aerosil®) were mixed and then sodium stearyl fumarate was added for final mixing, and the mixture was further compressed into tablet cores.

The tablet cores were then coated with a film coating material to produce film coated tablets. (Approximately 3% weight gain).

### Example 17

### Preparation of a Monolithic Tablet Containing Emtricitabine, Tenofovir, Darunavir and Ritonavir

| | |
|---|---|
| Raw Material | mg/tablet |

Darunavir Granulate

| | |
|---|---|
| Darunavir Hydrate | 865.75 |
| Hypromellose (Methocel ® E-15) | 12.20 |
| Croscarmellose Sodium (Ac-Di-Sol ®) | 60.99 |
| Purified Water | |

Emtricitabine/Tenofovir Granulate

| | |
|---|---|
| Emtricitabine | 200.0 |
| Tenofovir | 300.0 |
| Pregelatinized Starch | 31.8 |
| Microcrystalline cellulose (Avicel ® 101) | 25.0 |
| Croscarmellose Sodium (Ac-di-sol ®) | 25.0 |
| Purified water | |
| Ex-Granular (Part I) | |
| Microcrystalline cellulose (Avicel ® 102) | 239.08 |
| Crospovidone LX | 50.00 |
| Silicon di-oxide (Aerosil ®) | 6.0 |
| Croscarmellose Sodium (Ac-di-Sol ®) | 34.1 |
| Ritonavir Form II | 70.0 |
| Ex-Granular (Part II) | |
| Sodium stearyl fumarate | 29.30 |
| Total Tablet weight | 1949.22 |

Darunavir granulation: Darunavir, and Croscarmellose Sodium (Ac-Di-Sol) were wet granulated by top spray process using Hypromellose (Methocel® E-15) as granulation solution and purified water as granulation liquid.

The wet granulated material was then dried in Fluid bed drier and milled by Quadro to desired particle size.

Emtricitabine & Tenofovir granulation: Emtricitabine & Tenofovir were wet granulated with microcrystalline cellulose, pregelatinized starch & croscarmellose sodium in High shear mixer with purified water as granulation liquid.

The wet granulated material was then dried in Fluid bed drier and milled by Quadro to desired particle size.

Darunavir granulate, Emtricitabine & Tenofovir granulate, Ritonavir Form II, crospovidone, microcrystalline cellulose (Avicel 102), and Silicon di-oxide (Aerosil) were mixed and then sodium stearyl fumarate was added for final mixing, and the mixture was further compressed into tablet cores.

The tablet cores are then coated with a film coating material to produce film coated tablets. (Approximately 3% weight gain).

### Example 18

### Preparation of a Monolithic Tablet Containing Emtricitabine, Tenofovir, Darunavir and Ritonavir

| | |
|---|---|
| Raw Material | mg/tablet |

Darunavir Granulate

| | |
|---|---|
| Darunavir Hydrate | 865.75 |
| Hypromellose (Methocel ® E-15) | 12.20 |
| Croscarmellose Sodium (Ac-Di-Sol ®) | 60.99 |
| Purified Water | |

Emtricitabine/Tenofovir Granulate

| | |
|---|---|
| Emtricitabine | 200.0 |
| Tenofovir | 300.0 |
| Pregelatinized Starch | 31.8 |
| Microcrystalline cellulose (Avicel ® 101) | 25.0 |
| Croscarmellose Sodium (Ac-Di-sol ®) | 25.0 |
| Ritonavir Form II | 70.0 |
| Purified water | |
| Ex-Granular (Part I) | |
| Microcrystalline cellulose (Avicel ® 102) | 239.08 |
| Crospovidone LX | 50.00 |
| Silicon di-oxide (Aerosil ®) | 6.0 |
| Croscarmellose Sodium (Ac-Di-Sol ®) | 34.1 |
| Ex-Granular (Part II) | |
| Sodium stearyl fumarate | 29.30 |
| Total Tablet weight | 1949.22 |

Darunavir granulation: Darunavir, and Croscarmellose Sodium (Ac-Di-Sol®) were wet granulated by top spray process using Hypromellose (Methocel® E-15) as granulation solution and purified water as granulation liquid.

The wet granulated material was then dried in Fluid bed drier and milled by Quadro to desired particle size.

Emtricitabine, Tenofovir & Ritonavir granulation: Emtricitabine, Tenofovir & Ritonavir are wet granulated with microcrystalline cellulose, pregelatinized starch & croscarmellose sodium in High shear mixer with purified water as granulation liquid.

The wet granulated material was then dried in Fluid bed drier and milled by Quadro to desired particle size.

Darunavir granulate, Emtricitabine, Tenofovir & Ritonavir Form II granulate, crospovidone, microcrystalline cellulose (Avicel® 102), and Silicon di-oxide (Aerosil®) were mixed and then sodium stearyl fumarate was added for final mixing, and the mixture was further compressed into tablet cores.

The tablet cores are then coated with a film coating material to produce film coated tablets. (Approximately 3% weight gain).

What is claimed is:

1. A pharmaceutical formulation in a unit dosage form comprising:
(a) tenofovir or a physiologically functional derivative thereof;
(b) emtricitabine or a physiologically functional derivative thereof;
(c) darunavir or a physiologically functional derivative thereof; and
(d) ritonavir, wherein the amount of ritonavir is less than about 100 mg and the ritonavir is in the form of ritonavir crystalline form II.

2. A pharmaceutical formulation according to claim 1 wherein the tenofovir is in the form of its prodrug tenofovir disoproxil, or wherein the tenofovir disoproxil is in the form of its fumarate salt (tenofovir disoproxil fumarate).

3. A pharmaceutical formulation according to claim 1, wherein the darunavir is in the form of darunavir ethanolate, darunavir hydrate, or amorphous darunavir.

4. A pharmaceutical formulation according to claim 1 in the form of a solid unit dosage form.

5. A pharmaceutical formulation according to claim 1 in the form of a tablet.

6. A pharmaceutical formulation according to claim 1 in the form of a film-coated tablet.

7. A pharmaceutical formulation according to claim 1 wherein the pharmaceutical dosage form is a monolithic (single layer) tablet.

8. A pharmaceutical formulation according to claim 1 wherein when the pharmaceutical formulation is administered to a patient peak plasma concentrations of each of the active agents (a)-(c) are achieved which are comparable to peak plasma concentrations achieved by the administration of the active agents (a)-(c) as separate unit doses to the patient.

9. A pharmaceutical formulation according to claim 1 wherein when the pharmaceutical formulation is administered to a patient a peak plasma concentration of ritonavir is achieved which is less than that achieved by administration of the commercially available ritonavir tablet composition (Norvir®).

10. A pharmaceutical formulation according to claim 1 wherein the amount of ritonavir is less than or equal to about 90 mg.

11. A pharmaceutical formulation according to claim 1 wherein the amount of ritonavir is at least about 50 mg.

12. A pharmaceutical formulation according to claim 1 wherein the amount of ritonavir is less than or equal to about 70 mg.

13. A pharmaceutical formulation according to claim 1 wherein the pharmaceutical formulation is in the form of a tablet and the tablet has a total weight of between about 1.800 g to about 2.200 g.

14. A pharmaceutical formulation according to claim 1 wherein the pharmaceutical formulation is in the form of a tablet and the largest maximum diameter of the tablet is about 27 mm or less and the depth of the tablet is less than about 12.5 mm.

15. A pharmaceutical formulation according to claim 1 wherein the unit dosage form comprises about 60% to about 85% by weight of active agents (a)-(d).

16. A pharmaceutical formulation according to claim 1 wherein the unit dosage form is in the form of a monolithic (single layer) tablet comprising:
(a) about 300 mg of tenofovir disoproxil fumarate,
(b) about 200 mg of emtricitabine,
(c) about 800 mg of darunavir and
(d) less than or equal to about 90 mg of ritonavir.

17. A pharmaceutical formulation according to claim 16 wherein the tablet has a total weight of between about 1.800 g to about 2.200 g.

18. A pharmaceutical formulation according to claim 16 wherein the unit dosage form comprises about 60% to about 85% by weight of active agents (a)-(d).

19. A process for preparing a pharmaceutical formulation according to claim 1, wherein the pharmaceutical formulation is a a monolithic tablet, the process comprising:
(a) blending a mixture comprising tenofovir disoproxil (or tenofovir disoproxil fumarate), emtricitabine, darunavir (or darunavir hydrate), ritonavir form II and optionally one or more pharmaceutically acceptable excipients to obtain a blend;
(b) compressing the blend into tablet cores; and
(c) optionally, applying one or more coating layers over the tablet cores.

20. A method for treating HIV-1 infection comprising administering to a patient infected by HIV-1 a pharmaceutically effective amount of the pharmaceutical formulation according to claim 1.
